(19) United States
(12) Patent Application Publication
(10) Pub. No.: US 2022/0323546 A1
(43) Pub. Date: Oct. 13, 2022

(54) RECOMBINANT INTERFERON

(71) Applicants: ALTUM PHARMACEUTICALS INC., Vancouver (CA); NATIONAL RESEARCH COUNCIL OF CANADA, Ottawa (CA)

(72) Inventors: SCOTT RUDGE, BOULDER, CO (US); LUKE MASSON, QUEBEC (CA); MÉLANIE ARBOUR, QUEBEC (CA); JULIE STRUBLE, BOTHELL, WA (US); JENAE LUELLEN, BOULDER, CO (US)

(21) Appl. No.: 17/235,654

(22) Filed: Apr. 20, 2021

Related U.S. Application Data
(60) Provisional application No. 63/012,904, filed on Apr. 20, 2020.

(51) Int. Cl. A61K 38/21; A61K 9/00; A61P 31/12; A61K 47/18; A61K 47/26; A61K 47/02

(52) U.S. Cl. CPC A61K 38/212; A61K 9/0075; A61P 31/12; A61K 47/183; A61K 47/26; A61K 47/02

(57) ABSTRACT

Isolated, pure, soluble isoform free recombinant human interferon alpha 2b (rhIFN α-2b). Methods of making the soluble isoform free rhIFN α-2b. Stable compositions and formulations of isoform free rhIFN α-2b are for administration generally for treatment and prevention of viral infections. In aspects, to prevent and/or minimize symptom(s) of viral infections such as respiratory infection(s) in a subject, in aspects SARS-COV2 infection.

### CROSS-REFERENCE TO RELATED APPLICATIONS

This application is based on and claims priority to U.S. Provisional Application Ser. No. 63/012,904, filed on Apr. 20, 2020, which is incorporated herein by reference in its entirety.

### INCORPORATION OF SEQUENCE LISTING

A paper copy of the Sequence Listing and a computer readable form of the Sequence Listing containing the file named “3522856.0002_ST25.txt”, which is 13,488 bytes in size (as measured in MICROSOFT WINDOWS® EXPLORER), are provided herein and are herein incorporated by reference. This Sequence Listing consists of SEQ ID NOs:1-15.

### FIELD

The invention relates to recombinant authentic interferon (IFN) that is isotypically pure. The invention further relates to methods for making recombinant authentic isotypically pure interferon alpha 2b, stable compositions and formulations thereof. Formulations have use for minimizing, preventing and/or for treatment of viral infections, in aspects viral respiratory infections.

### BACKGROUND

Pandemics have occurred throughout history and appear to be increasing in frequency, perhaps partly due to the increasing emergence of viral disease from animals coupled with the increase in global travel. Pandemic risk is driven by the combined effects of spark risk (where a pandemic is likely to arise) and spread risk (how likely it is to diffuse broadly through human populations).

Respiratory viruses in particular have pandemic potential as they spread via air borne droplets generated through coughing, sneezing heavy breathing. These viruses can also be spread by touching and transfer of the virus from contaminated objects to the nose and/or eyes potentially developing into disease.

Interferon medicaments having antiviral activity as yet have unfulfilled clinical promise to treat emerging viral respiratory infections. Some attempted solutions have been evaluated, however, due to unspecific antiviral effects definitive treatment cannot be sufficiently addressed owing to instability and inconclusive effects.

### SUMMARY

Type I interferons are known to have antiviral, anti-proliferative and immunomodulatory activities and have been used for treatment of certain cancers and viral infections via systemic or intramuscular administration. Oral administration to date has provided inconsistent outcomes. Currently, formulations of interferon alpha-2b (IFN α-2b) are heterogeneous despite exhaustive purification protocols and may demonstrate varying levels of immunogenicity and efficacy. The exhaustive purification protocols may also negatively affect protein conformation.

The present invention provides an improvement over currently available forms of therapeutic heterogeneous/mosaic recombinant alpha interferon that contain mixtures of interferon isoforms. Even small levels of interferon heterogeneity in a therapeutic may create stability issues and undesired physiological effects. Providing an isoform free (i.e. authentic single isotype) recombinant human IFN α-2b increases target specificity and reduces more generalized local and systemic undesired side effects. Biological activity may be enhanced.

The present invention provides a novel method for synthesizing an isoform free (i.e. authentic single isotype) recombinant human IFN α-2b (rhIFN α-2b).

The present invention also provides isoform free rh IFN α-2b as formulations that are surprisingly stable for extended time periods at a wide range of temperatures. The formulations remain stable meaning the isoform free rhIFN α-2b is not degraded such that it retains biological activity. Formulations remain clear, colorless, without microbial contamination for at least several months at temperatures of freezing, refrigeration and room temperatures and further with several cycles of freezing and thawing. Stability of the isoform free recombinant human IFN α-2b within therapeutic formulations for the treatment of viral infections, in aspects, viral respiratory infections is important to withstand manufacturing processes, storage and for presentation in devices for oral and/or nasal administration.

Severe acute respiratory syndrome coronavirus 2 (SARS-CoV-2) is highly contagious and causes coronavirus disease 2019 (COVID-19). COVID-19 rapidly became a worldwide pandemic. It can lead to acute respiratory distress syndrome (ARDS) triggering systemic multi-organ collapse and even death. Long lasting negative effects may affect several organs including the lungs, heart, kidneys and brain. Treatments are urgently required to minimize and/or prevent SARS-CoV-2 infection and minimize COVID-19 damage to lung and other tissues as well as prevent mortality. The isoform free recombinant human IFN-α 2b of the invention can be formulated for the targeted treatment of any variety of respiratory viral infections, in aspects to treat COVID-19.

In an aspect, presently provided is a recombinant (engineered) authentic interferon alpha 2b protein (IFN α-2b). In aspects a recombinant authentic human IFN α-2b protein (rhIFN α-2b). By “authentic” is meant that the rhIFN α-2b is a single isotype and free of other undesired isoforms, and is made as a substantially homogenous protein. The production of non-natural isoforms resulting from recombinant expression is prevented. The rhIFN-α 2b is thus pure, free of undesired isoforms. In aspects over 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or 100% pure.

In aspects are compositions comprising the authentic rhIFN α-2b.

In aspects are stabilized formulations comprising the authentic rhIFN α-2b. In aspects, is a method for making stabilized formulations comprising the authentic rhIFN α-2b.

In an aspect, the rhIFN α-2b may be pegylated. In aspects, fused with human serum albumin (HAS).

In aspects, is a novel method for synthesizing isoform free rhIFN α-2b.

The method incorporates a novel construct transformed into E. coli for high yield synthesis of recombinant human IFN α-2b while simultaneously preventing the formation of undesired isoforms of IFN α-2b. In this manner only recombinant authentic isoform free human IFN α-2b is synthesized.

In aspects, the recombinant human IFN-α 2b described herein has higher affinity for receptor binding. The increased specificity of the recombinant human IFN-α 2b described herein comes from making a pure form of human IFN α-2b that is isoform free. The method of the invention prevents the formation of IFN α-2b isoforms and therefore complex purification steps to separate isoforms are not required. This provides a higher affinity due to the homogeneity of the IFN α-2b and biophysical stability.

The recombinant human IFN α-2b of the invention is provided as a formulation demonstrated to be substantially more stable at room temperature, at refrigeration temperatures and at freezing storage temperatures for several weeks and up to several months compared to conventional heterogeneous IFN α-2b injection compositions. Formulations of recombinant human IFN α-2b of the invention are demonstrated to remain stable after multiple freeze-thaw cycles, for example 7 to 9 freeze-thaw cycles.

Recombinant isotype pure unmodified human interferon α-2b exhibits subtle differences in dynamics when compared with heterogeneous human interferon α-2b thus making it a more desired candidate for targeted more effective uses in the treatment of viral respiratory disorders.

The invention provides several embodiments including but not limited to: stable recombinant isotypically pure unmodified human interferon α-2b; stable recombinant isotypically pure pegylated human interferon α-2b; method for making the stable recombinant isotypically pure unmodified human interferon α-2b; method for making soluble stable recombinant recombinant isotypically pure unmodified human interferon α-2b; plasmids/constructs encoding the stable recombinant isotypically pure unmodified human interferon α-2b; producers (various E. coli hosts) comprising the plasmids/constructs and expressing the stable recombinant isotypically pure unmodified human interferon α-2b; fusion sequences coding for the synthesis of the stable recombinant isotypically pure unmodified human interferon α-2b; wet and dry compositions of stable recombinant isotypically pure unmodified human interferon α-2b; wet and dry formulations comprising the stable recombinant isotypically pure unmodified human interferon α-2b; methods of treatment of viral infection by administration of formulations of stable recombinant isotypically pure unmodified human interferon α-2b; methods of treatment of viral respiratory infection by administration of formulations of stable recombinant isotypically pure unmodified human interferon α-2b; methods of treatment of COVID19 by administration of formulations of stable recombinant isotypically pure unmodified human interferon α-2b; aerosol, mist, gel, cream, nebulized droplets and spray formulations of stable recombinant isotypically pure unmodified human interferon α-2b; methods of treatment of viral respiratory infection in patients by administration of aerosol, mist, gel, nebulized droplets and spray formulations of stable recombinant isotypically pure unmodified human interferon α-2b; methods of minimizing symptoms of viral respiratory infection in patients by administration of aerosol, mist, gel, nebulized droplets and spray formulations of stable recombinant isotypically pure unmodified human interferon α-2b; methods of prevention of viral respiratory infection in patients by administration of aerosol, mist, gel, nebulized droplets and spray formulations of stable recombinant isotypically pure unmodified human interferon α-2b; methods of direct lung administration via inhalation and methods for nasal administration of aerosol, mist, gel, droplets and spray formulations of stable recombinant isotypically pure unmodified human interferon α-2b; devices comprising isotypically pure unmodified human interferon α-2b for delivery of aerosol, mist, gel, nebulized droplets and spray formulations of the stable recombinant isotypically pure unmodified human interferon α-2b; kits comprising stable recombinant isotypically pure unmodified human interferon α-2b compositions or formulations; kits comprising devices loaded with stable recombinant isotypically pure unmodified human interferon α-2b and ready for use; kits comprising devices with dosages of stable recombinant isotypically pure unmodified human interferon α2b and ready for use, and kits comprising devices loaded with stable recombinant isotypically pure unmodified human interferon α-2b and one or more other active and ready for use.

According to an aspect of the invention is an efficient E. coli based expression system for synthesizing human IFN α-2b that is isoform free, wherein the human IFN-α-2b is expressed as a fusion protein comprising a cleavable protective tag. The cleavable tag serves the multiple roles to separate an N-terminal methionine in proximity to the N-terminal cysteine, to prevent E. coli modification of the N-terminal cysteine, to increase protein solubility, to increase protein expression and for enabling one-step or two-step purification of the fusion, in aspects by immobilized metal affinity chromatography (IMAC).

In aspects the cleavable protective tag is His-tagged ubiquitin. In aspects the cleavable protective tag is thioredoxin. In further aspects a linker can be used immediately upstream to the N-terminal cysteine and serve as a cleavage site. Suitable linker sequences may be selected from the group consisting of GSGSGDDDDK (SEQ ID NO: 5), GSEQ (SEQ ID NO: 6), GSDEE (SEQ ID NO: 7), GSEEEDDDG (SEQ ID NO: 8), GSEEEDDDGKK (SEQ ID NO: 9), GSEQKGGGEEDDG (SEQ ID NO: 10), GSEEDDDEEK (SEQ ID NO: 11), GSEQKGGGEEDDEE (SEQ ID NO: 12), and GSEQKGGGDEDG (SEQ ID NO: 13). In aspects, the linker sequence is immediately upstream to the C-terminal cysteine. Sequences similar to these linker sequences may be used herein.

In the method of the invention, the human IFN α-2b expressed fusion is purified from E. coli extracts by affinity adsorption, for example to an IMAC resin, after which enzymatic or chemical cleaving is performed, followed by a final purification step using IMAC to isolate the leader sequence and uncleaved product. Other additional purification steps are optional. The cleaved product is subjected to N-terminal sequencing confirming the identical sequence to that expected for the amino acid sequence of human IFN α-2b. Isoforms of IFN-α 2b are not present.

In accordance with an aspect of the invention is a vector construct comprising a promoter, a nucleotide sequence encoding IFN α-2b and a cleavable protective tag at the 3′ end of the open reading frame. The vector may further comprise a linker immediately upstream to the desired N-terminal cysteine.

In accordance with an aspect of the invention is a nucleic acid sequence encoding a human IFN α-2b fusion protein. In aspects is a vector comprising the nucleic acid sequence. In aspects is a host cell expressing the nucleic acid sequence, wherein the host cell is a strain of E. coli.

In accordance with an aspect of the invention is IFN α-2b fusion protein comprising an amino acid sequence of SEQ ID NO.1.

In accordance with an aspect of the invention is IFN α-2b fusion protein comprising or consisting of the amino acid sequence of any one of SEQ ID NO.2, SEQ ID NO.3 or SEQ ID NO.4.

In an aspect is an amino acid sequence encoding human IFN α-2b, wherein the amino acid sequence further comprises an N-terminal protection tag. In aspects the N-terminal protection tag prevents modification of the N-terminal cysteine from modification during expression in E. coli.

According to an aspect of the invention is an amino acid sequence of any one of SEQ ID NO.2, SEQ ID NO.3 or SEQ ID NO.4.

According to an aspect of the invention is an amino acid sequence of any one of SEQ ID NO.2, SEQ ID NO.3 or SEQ ID NO.4, wherein said amino acid sequence binds to a metal. In aspects the metal is nickel.

According to another aspect of the invention is an amino acid sequence of any one of SEQ ID NO.2, SEQ ID NO.3 or SEQ ID NO.4 for expressing human IFN α-2b. In aspects, any one of SEQ ID NO.2, SEQ ID NO.3 or SEQ ID NO.4 comprises an enzyme cleavage site to remove/cleave the human IFN α-2b.

According to an aspect of the invention is a composition comprising or consisting of the polypeptide of SEQ ID NO.2, SEQ ID NO.3 or SEQ ID NO.4.

According to an aspect of the invention is a nucleic acid sequence encoding the polypeptide of SEQ ID NO.2, SEQ ID NO.3 or SEQ ID NO.4.

The invention includes sequences that share at least 80%, at least 85%, at least 90%, at least 95%, at least 96%, at least 97%, at least 98%, or at least 99% or more sequence identity to the sequence of any one of SEQ ID NO.2, SEQ ID NO.3 or SEQ ID NO.4.

The invention includes sequences that share at least 80% or more sequence identity to the sequence of any one of SEQ ID NO.2, SEQ ID NO.3 or SEQ ID NO.4, excluding the human IFN α-2b sequence.

In aspects, is a recombinant authentic (isoform free) interferon composition. In aspects, the interferon is type I interferon, in further aspects is IFN-α, in further aspects is human IFN-α 2b, in further aspects pegylated human IFN-α 2b. The recombinant authentic (isoform free) interferon has antiviral activity and increased biological activity relative to presently reported commercially sourced heterogeneous IFN α-2b formulations and is advantageously stable compared to commercially sourced heterogeneous IFN α-2b formulations.

Recombinant authentic (isoform free) IFN α-2b formulations of the invention have surprisingly increased stability compared with heterogenous IFN α-2b formulations. In aspects the formulations comprise one or more stabilizing agents that help to stabilize the isoform free human IFN-α 2b within the formulation to prevent and/or minimize oxidation thereof.

The recombinant isoform free human IFN α-2b eluted protein can be lyophilized and stored at freezing temperatures ranging from less than 0° C., for example from −20° C. to −80° C. for up to several years; at refrigeration temperatures of about 2° C. to about 4° C. for several months up to about 1 year, or up to about 2 years or more; or at room temperatures (about 20° C. to about 25° C.) for up to about a year. The isoform free rhIFN-α 2b can be reconstituted for use with sterile water and/or buffer(s), and formulated for therapeutic use remaining stable at varying temperatures for weeks for up to 6 months or more.

The recombinant isoform free IFN-α 2b eluted protein can be stored in desired aliquots at temperatures ranging from for example at −20° C. to −80° C. for up to several years; at refrigeration temperatures of about 2° C.-8° C. (+/−2° C.) for at least 6 months; or at room temperatures (about 20° C.+/−2° C. to about 25° C.+/−2° C.) for at least 6 months. Alternatively, the eluted protein can be lyophilized and stored.

In an aspect of the invention is frozen recombinant isoform free hIFN-α 2b. In further aspects, a frozen stable formulation of recombinant isoform free hIFN-α 2b, in further aspects aliquots of frozen recombinant isoform free hIFN-α 2b.

The recombinant isoform free IFN α-2b compositions are formulated for administration for the treatment and/or prevention and/or minimizing symptoms of respiratory infections caused by a respiratory virus. In aspects the respiratory virus is a coronavirus (alpha, beta, gamma and delta) such as human coronaviruses: 229E (alpha coronavirus); NL63 (alpha coronavirus); OC43 (beta coronavirus); HKU1 (beta coronavirus); MERS-CoV (beta coronavirus causing Middle East respiratory syndrome); SARS-CoV (beta coronavirus causing severe acute respiratory syndrome); and SARS-CoV-2 (new coronavirus causing COVID-19).

In further aspects, the respiratory virus is: an influenza virus (for example, swine flu H1N1 influenza-A virus; avian flu H5N1 influenza-A virus; seasonal influenza A virus, and seasonal influenza B virus); parainfluenza virus; adenovirus including 57 types leading to the common cold; respiratory syncytial virus (RSV), rhinovirus or meta-pneumovirus.

In further aspects, formulations of the recombinant isoform free IFN α-2b is helpful for treatment of viral pneumonia that may develop from other earlier viral infection from adenoviruses, varicella zoster virus, influenza viruses and RSV.

According to an aspect of the invention is a recombinant isoform free human IFN α-2b stable formulation for administration to a patient for the treatment of COVID-19 in a patient. According to a further aspect of the invention is a recombinant isoform free human IFN α-2b stable formulation for administration to a subject to prevent and/or minimize risk of infection by SARS-CoV-2.

According to a further aspect of the invention is a recombinant isoform free IFN α-2b composition formulated for administration to a subject to minimize one or more symptoms of infection by SARS-CoV-2.

In a further aspect, recombinant isoform free IFN α-2b compositions are formulated for administration to a patient for the treatment of viral respiratory infections, in aspects COVID-19. Such formulations are also for improving and/or ameliorating one or more undesired symptoms of a viral respiratory infection such as COVID-19. Furthermore, formulations are also for preventing infection by a respiratory virus, in aspects inhibit SARS-COV-2 infection and thus the development of COVID-19.

Formulations can be for pulmonary (lung) administration or nasal administration. The formulations can be wet or dry. The formulations can be reconstituted wet formulations. Dry formulations can be provided with excipients for administration of a dry powder aerosol of the recombinant isoform free IFN-α 2b of the invention. Wet formulations can be provided with suitable excipients and/or solvents for administration of liquid aerosol, liquid spray, or nebulized droplets for inhalation and/or for nasal breathing. The formulation can be presented as a gel for nasal administration.

Localized lung administration reduces deleterious long term effects of treatment. Localized administration is desired to reach viral disease in the superficial cells of the respiratory airway lumen, where immune protection has reduced effectiveness.

Localized administration via nasal administration can prevent virus, such as for example SARS-COV-2, from invading the nasal mucosa and thus stop/minimize infection. Localized nasal administration also helps to prevent a minor localized nasal mucosal viral infection from spreading and reaching the respiratory tract. Nasal administration is suitable at low doses with frequency as a preventative measure against infection.

Formulations of the invention are self-administrable or for administration.

Formulations of the invention can be administered in a single dose or in several doses per day in accordance with a schedule, and for several days.

Formulations of the invention are intended for administration at any first sign/symptom of possible COVID19 infection which may include one or more of: fever (sudden or persistent); pink eye; feeling tired/exhaustion; aching body; dry cough; sweating; confusion; sore throat; headache; difficulty sleeping; loss of appetite; diarrhea; gastrointestinal pain; anosmia; trouble breathing; rapid breathing; dyspnea; dizziness; nasal congestion; chills, bluish lips; and general sense of unwellness. Administration of formulations of the invention at the earliest sign of any one or more symptom, at times of the most possible early infection, may provide the best treatment outcome as the localized administration of recombinant isoform free IFN α-2b can prevent and/or slow down SARS-COV-2 virus replication and thus movement into the lungs. The formulations of the invention may act to bypass interferon blockage in cells and restore IFN inhibition of viral replication to support a normal immune response.

Formulations of the invention are also intended for administration to subjects at increased risk of contacting an infectious respiratory virus or increased risk for severity of disease by virtue of age, having certain underlying medical conditions, or being in a higher risk atmosphere (for example, a first responder, a hospital worker and the like) as a precaution to minimize their own risk of infection.

The recombinant isoform free human IFN α-2b is formulated to comprise one or more stabilizing agents for stabilization of the recombinant isoform free human IFN α-2b thus extending its biological activity and extending its shelf life. In an aspect the stabilizing agent functions for example as an antioxidant that can prevent de-oxidation or dimerization of cysteine (protein kept conformationally stable.) Suitable anti-oxidant stabilizing agents are water soluble and selected from the group consisting of methionine, cysteine, vitamin C (D-ascorbic acid/sodium ascorbate), glutathione, lipoic acid, uric acid and combinations thereof. In aspects the stabilizing agent is methionine. In aspects the stabilizing agent is lipoic acid.

The recombinant isoform free IFN-α 2b formulations of the invention may further comprise one or more pharmaceutically acceptable carriers/excipients and may further comprise one or more additional antiviral agent(s).

Provided herein are methods to provide lung-targeted therapies for patients with a viral respiratory infection such as COVID19 wherein an isoform free rhIFN α-2b composition is formulation for delivery to the pulmonary vascular bed with minimal off-target exposure via a portable device. The inhaled formulations provided herein are characterized by good pulmonary tolerance and little or no gastrointestinal or systemic exposure, making them particularly advantageous for use.

Provided herein are methods to provide nasal-targeted therapies for a subject at risk of SARS-COV-2 infection wherein an isoform free IFN α-2b formulation is delivered via nasal inhalation to the nasal mucosa with minimal off-target exposure via a portable device. The nasal inhalation formulations provided herein are characterized by good tolerance and little or no gastrointestinal or systemic exposure, making them particularly advantageous for use.

According to an aspect of the invention is a method for treatment of a respiratory viral infection in a patient comprising administering a therapeutically effective amount of an isoform free human IFN α-2b formulation to the lungs of the patient, wherein the formulation is an aerosol, nebulized droplets or a spray.

According to an aspect of the invention is a pressurized metered dose inhaler (pMDI), dry powder inhaler (DPI), soft mist inhaler (SMI), nebulizer device or spray bottle provided as a ready to use device comprising one or more doses of an isoform free human IFN α-2b stable formulation of the invention.

Aspects of the invention are as follows:
1. Isoform free recombinant interferon alpha 2b (rIFN α-2b).
2. Human isoform free recombinant interferon alpha 2b (rhIFN α-2b).
3. Isolated isoform free recombinant interferon alpha 2b (rhIFN α-2b).
4. The isoform free rhIFN α-2b of any one of claims 1 to 3, wherein the recombinant rhIFN α-2b is at least 99% pure, at least 99.5% pure or 100% pure with respect to degradation isoforms.
5. The isoform free rhIFN α-2b of any one of claims 1 to 4, wherein the N-terminal of the rhIFN α-2b protein is homogenous.
6. The isoform free rhIFN α-2b of any one of claims 1 to 5, wherein the rhIFN α-2b exhibits a stable confirmation relating to to oxidative protein folding of cysteine residue(s).
7. The isoform free rhIFN α-2b of any one of claims 1 to 6, wherein the rhIFN α-2b has a high specific activity.
8. The isoform free recombinant interferon alpha 2b of any one of claims 1 to 7, wherein the isoform free rhIFN α-2b is soluble.
9. A composition consisting of the isoform free rhIFN α-2b of any one of claims 1 to 8.
10. A composition comprising the isoform free rhIFN α-2b of any one of claims 1 to 5.
11. The composition of claim 9 or 10, wherein the composition is sterile.
12. The composition of any one of claims 9 to 11, comprising an acceptable carrier, excipient, diluent or mixtures thereof.
13. A formulation comprising the composition of any one of claims 9 to 12, formulated with an agent for stabilizing the rhIFN α-2b.
14. The formulation of claim 13, wherein the agent functions as an antioxidant to prevent/minimize de-oxidation of the rhIFN α-2b to maintain folded conformation.
15. The formulation of claim 13 or 14, wherein the agent is water soluble and selected from the group consisting of methionine, cysteine, vitamin C (D-ascorbic acid), glutathione, lipoic acid, uric acid and combinations thereof.
16. The formulation of claim 15, wherein the agent is methionine.
17. The formulation of claim 16, wherein methionine is present in an amount of up to about 5 mg/ml.
18. The formulation of claim 16, wherein methionine is present in an amount of up to about 1.0 mg/ml, up to about 1.5 mg/ml, about 1.8 mg/ml, up to about 2.0 mg/ml, up to about 2.5 mg/ml, up to about 3.0 mg/ml, up to about 3.5 mg/ml, up to about 4.0 mg/ml, up to about 4.5 mg/ml, and about 5.0 mg/ml.
19. The formulation of claim 15, wherein the agent is lipoic acid.
20. The formulation of claim 19, wherein the lipoic acid is present in an amount of up to about 1.0 mg/ml, up to about 1.5 mg/ml, about 1.8 mg/ml, up to about 2.0 mg/ml, about 2.2 mg/ml, up to about 2.5 mg/ml, up to about 3.0 mg/ml, up to about 3.5 mg/ml, up to about 4.0 mg/ml, up to about 4.5 mg/ml, and about 5.0 mg/ml.
21. The formulation of any one of claims 9 to 20, wherein the recombinant isoform free IFN-α 2b is provided up to about 10 mg/ml, or up to about 15 mg/ml.
22. The formulation of any one of claims 9 to 20, wherein the recombinant isoform free IFN-α 2b is provided at about 10 μg/ml to about 400 μg/ml.
23. The formulation of any one of claims 4 to 15, wherein the recombinant isoform free IFN-α 2b is provided at about 5.0 MIU/ml.
24. The formulation of any one of claims 9 to 23, wherein the formulation is made from a reconstituted lyophilized composition of any one of claims 9 to 12.
25. The formulation of claim 24, wherein the formulation comprises a desired concentration of recombinant isoform free IFN-α 2b per ml.
26. The formulation of any one of claims 9 to 25, wherein the formulation is stable at temperatures of −80° C. to about 40° C.
27. The formulation of claim 26, wherein the formulation is stable for up to 1 about month, up to about 2 months, up to about 3 months, up to about 6 months, up to about 1 year and up to about 2 years.
28. The formulation of any one of claims 13 to 27, formulated as a liquid for administration to a subject via inhalation, intravenous, intramuscular, intrathecal, parenteral or intravaginal.
29. The formulation of claim 28, formulated for inhalation as a liquid aerosol, liquid droplets, liquid mist, or as a liquid spray.
30. The formulation of claim 29, for inhalation into the lungs or nasal inhalation.
31. The formulation of any one of claims 28 to 30, in conjunction with a metered dose inhaler (MDI), soft mist inhaler (SMI), nebulizer, or spray bottle.
32. The formulation of claim 28, formulated for intravaginal administration as a an aerosol, as droplets, as a mist, as a spray, as a gel or as a creme.
33. The formulation of any one of claims 13 to 27, formulated for inhalation as a powder aerosol, powder mist, or powder particulate spray.
34. The formulation of claim 33, wherein the powder comprises particles of up to about 5 μm.
35. The formulation of claim 33 or 34, provided in conjunction with a dry powder inhaler (DPI).
36. The formulation of any one of claims 13 to 27, further comprising or used in conjunction with one or more of an antiviral selected from Remdesivir, lopinavir, Favipiravir, Darunavir, Oseltamivir, Umifenovir, Novaferon; immune modulating drug selected from immunoglobulins, monoclonal antibodies; glucocorticoid; anti-inflammatory drug selected from leflunomide, colchicine, naproxen, piclidenoson; cardiovascular drug selected from ACE-2, ACE-inhibitor, ARB, angiotension; Chloroquine; Hydroxychloroquine; Aviptadil, Azithromycin; Itraconazole; Vitamin D; and zinc.
36. A method of treating a viral pulmonary infection in a subject, comprising administering by inhalation to the respiratory tract of the subject, an effective amount of the formulation of any one of claims 13 to 35.
37. The method of claim 36, wherein the viral respiratory infection is caused by a virus selected from a coronavirus (alpha, beta, gamma and delta); human coronaviruses 229E (alpha coronavirus), NL63 (alpha coronavirus), OC43 (beta coronavirus), and HKU1 (beta coronavirus); MERS-CoV (beta coronavirus causing Middle East respiratory syndrome); SARS-CoV (beta coronavirus causing severe acute respiratory syndrome); and SARS-CoV-2 (new coronavirus causing COVID-19 disease).
38. The method of claim 37, wherein the virus is SARS-CoV-2 causing COVID-19 disease.
39. The method of claim 38, wherein said formulation is provided at a dose of about 5 MIU/ml for twice daily administration for up to about 14 days.
40. The method of claim 38 or 39, wherein the formulation is for administration to the subject with a first symptom of COVID-19 disease.
41. The method of claim 38 or 39, wherein the formulation is for administration to the subject prior to a first symptom of COVID-19 disease.
42. The method of claim 38 or 39, wherein the formulation is for administration to the subject at risk of infection with SARS-CoV-2.
43. The method of claim 36, wherein the viral respiratory infection is caused by a virus selected from an influenza virus (swine flu H1N1 influenza-A virus; avian flu H5N1 influenza-A virus; seasonal influenza A or B) a parainfluenza virus, an adenovirus (common cold), respiratory syncytial virus (RSV), a rhinovirus or a meta-pneumovirus.
44. A method of treating a viral infection in a subject, comprising administering to the subject, an effective amount of the formulation of any one of claims 13 to 35, wherein the viral infection is caused by: Flaviviruses selected from Dengue, Zika and West Nile virus; Filoviruses selected from Ebola; Bunyaviruses selected from Hantavirus; and Hepdna viruses selected from Hepatitis A, Hepatitis B and Hepatitis C.
45. A method for treating a patient with COVID-19 comprising administering an aerosol formulation comprising the formulation of any one of claims 13 to 35 to the patient.
46. A method for prevention of SARS-COV-2 viral infection in a subject at risk for infection, the method comprising administering an aerosol comprising the formulation of any one of claims 13 to 35 to the subject.
47. A method for prevention of SARS-COV-2 viral infection in a subject at risk of infection, the method comprising nasal administration of an aerosol formulation of claim 30 to the subject.
48. A method for treatment of a respiratory viral infection in a patient comprising administering a therapeutically effective amount of an isoform free recombinant human IFN-α 2b stable formulation to the lungs of the patient, wherein the formulation is an aerosol, nebulized droplets or a spray.
49. A stable formulation of recombinant isoform free human IFN-α 2b (rh IFN-α 2b), the formulation comprising:
   about 10 μg/ml to about 400 μg/ml recombinant isoform free IFN-α 2b;
   up to about 5 mg/ml methionine;
   Polysorbate 80;
   EDTA; and
   sodium phosphate buffer,
   wherein said formulation is stable at temperatures ranging from about −80° C. to about 40° C.
50. The stable aqueous formulation of IFN-α 2b of claim 49, wherein said formulation is stable at temperatures of about 2° C. to about 40° C. for up to six months.
51. A nebulizer comprising the stable formulation of claim 49 or 50.
52. The stable formulation of claim 49 or 50, formulated as a stable dry powder comprising particles of up to about 5 μm.
53. The stable formulation of claim 52, wherein the dry powder comprises a carrier powder.
54. A dry powder dose inhaler or metered dose inhaler comprising the stable formulation of claim 52 or 53.
55. Use of the isoform free recombinant interferon alpha 2b of any one of claims 1 to 8 to make a medicament for the treatment of a viral infection.
56. Use of the isoform free recombinant interferon alpha 2b of any one of claims 1 to 8 to make a medicament for the treatment of a respiratory infection.
57. Use of the pure isoform free recombinant interferon alpha 2b of any one of claims 1 to 8 or the formulation of any one of claims 13 to 36 for the treatment or prophylaxis of a viral respiratory infection in a subject.
58. An efficient E. coli expression system that expresses isoform free human IFN-α 2b.
59. The E. coli expression system of claim 58, wherein the IFN-α 2b is expressed as a fusion to a cleavable protective tag.
60. The E. coli expression system of claim 59, wherein cleavable protective tag protects the N-terminal cysteine from modification.
61. The E. coli expression system of claim 60, wherein prevention of the E. coli modification of the N-terminal cysteine increases solubility, increases expression and enables one-step purification of the fusion.
62. The E. coli expression system of claim 61, wherein the purification comprises immobilized metal affinity chromatography (IMAC).
63. The E. coli expression system of any one of claims 58 to 62, wherein the cleavable protective tag is His-tagged ubiquitin.
64. The E. coli expression system of any one of claims 58 to 62, wherein the cleavable protective tag is thioredoxin.
65. The E. coli expression system of any one of claims 58 to 64, comprising a linker inserted immediately upstream to the N-terminal cysteine to serve as a cleavage site.
66. The E. coli expression system of claim 65, wherein the linker sequence is selected from the group consisting of GSGSGDDDDK (SEQ ID NO: 5), GSEQ (SEQ ID NO: 6), GSDEE (SEQ ID NO: 7), GSEEEDDDG (SEQ ID NO: 8), GSEEEDDDGKK (SEQ ID NO: 9), GSEQKGGGEEDDG (SEQ ID NO: 10), GSEEDDDEEK (SEQ ID NO: 11), GSEQKGGGEEDDEE (SEQ ID NO: 12), and GSEQKGGGDEDG (SEQ ID NO: 13).
67. The E. coli expression system of claim 65 or 66, wherein the linker sequence is immediately upstream to the C-terminal cysteine.
68. A method for expressing a recombinant isoform free human IFN-α 2b comprising:

transfecting a host with a plasmid comprising an inducible promoter, a nucleotide sequence encoding human IFN-α 2b and a cleavable protective tag at the 3′ end of the open reading frame,

the host expressing the IFN-α 2b protein as a fusion;

purifying the fusion from the host extract by affinity adsorption and enzymatic or chemical cleaving; and

performing IMAC purification step to isolate the leader sequence and uncleaved product, wherein the product is pure isoform free human IFN-α 2b.
69. The method of claim 68, wherein the cleavable protective tag is directly downstream the N-terminal methionine.
70. The method of claim 68 or 69, wherein the isoform free human IFN-α 2b product is homogeneous at the N-terminal.
71. A vector construct comprising a promoter, a nucleotide sequence encoding human IFN-α 2b and a cleavable tag at the 3′ end of the open reading frame.
72. The vector construct of claim 71, comprising a linker immediately upstream the N-terminal cysteine.
73. A nucleic acid sequence encoding an isoform free human IFN-α 2b fusion protein.
74. A vector comprising the nucleic acid sequence of claim 73.
75. A host cell comprising the vector of claim 74, wherein the host cell is a strain of E. coli.
76. An isoform free recombinant human IFN-α 2b fusion protein comprising or consisting of the amino acid sequence of any one of SEQ ID NO.2, SEQ ID NO.3 and SEQ ID NO.4.
77. An amino acid sequence encoding isoform free human IFN-α 2b, wherein the amino acid sequence further comprises an N-terminal protection tag immediately downstream the N-terminal methionine, wherein the N-terminal protection tag prevents modification of the N-terminal cysteine from modification during expression in E. coli.
78. An amino acid sequence of any one of SEQ ID NO.2, SEQ ID NO.3 or SEQ ID NO.4.
79. An amino acid sequence of any one of SEQ ID NO.2, SEQ ID NO.3 or SEQ ID NO.4, wherein said amino acid sequence binds to a metal.
80. An amino acid sequence of any one of SEQ ID NO.2, SEQ ID NO.3 or SEQ ID NO.4 encoding isoform free human IFN-α 2b and comprising an enzyme cleavage site to remove the human IFN-α 2b.
81. A composition comprising or consisting of the polypeptide of SEQ ID NO.2, SEQ ID NO.3 or SEQ ID NO.4.
82. A nucleic acid sequence encoding the polypeptide of SEQ ID NO.2, SEQ ID NO.3 or SEQ ID NO.4.
83. A recombinant isoform free human interferon alpha 2b having antiviral activity and increased biological activity relative to heterogeneous IFN-α 2b.
84. A recombinant isoform free human interferon alpha 2b formulated to be stable for several months at temperatures ranging from about 2° C. to about 40° C. relative to heterogeneous IFN-α 2b.
85. A formulation of isoform free human interferon alpha 2b comprising up to about 10 mg/ml rh IFN-α 2b, about 7.5 mg/ml NaCl, about 1.8 mg/ml sodium phosphate dibasic, about 1.3 mg/ml sodium phosphate monobasic, about 0.1 mg/ml EDTA, about 0.1 mg/ml Polysorbate 80 and about 2.2 mg/ml lipoic acid.
86. A formulation of isoform free human interferon alpha 2b comprising up to about 10 mg/ml rh IFN-α 2b, about 7.5 mg/ml NaCl, about 0.1 mg/ml EDTA, about 0.1 mg/ml Polysorbate 80 and about 3.5 mg/ml methionine.
87. A formulation of isoform free human interferon alpha 2b comprising up to about 10 mg/ml rh IFN-α 2b, about 7.5 mg/ml NaCl, about 0.9 mg/ml sodium phosphate dibasic, about 0.7 mg/ml sodium phosphate monobasic, about 0.1 mg/ml EDTA, about 0.1 mg/ml Polysorbate 80 and about 1.8 mg/ml methionine.
88. A formulation of isoform free human interferon alpha 2b comprising up to about 10 mg/ml rh IFN-α 2b, up to about 10 mg/ml NaCl, up to about 5.0 mg/ml sodium phosphate dibasic, up to about 5.0 mg/ml sodium phosphate monobasic, about to about 3.0 mg/ml EDTA, up to about 3.0 mg/ml Polysorbate 80 and up to about 4.0 mg/ml lipoic acid, wherein the formulation has a pH of about 7.2 to about 7.8 and is stable at freezing, refrigerated and room storage temperatures for at least several months.
89. A formulation of isoform free human interferon alpha 2b comprising up to about 10 mg/ml rh IFN-α 2b, up to about 10.0 mg/ml NaCl, up to about 4.0 mg/ml EDTA, up to about 3.0 mg/ml Polysorbate 80 and up to about 5.0 mg/ml methionine, wherein the formulation has a pH of about 7.2 to about 7.8 and is stable at freezing, refrigerated and room storage temperatures for at least several months.

### FIGURES

The above and other aspects, advantages, and features of this disclosure will become more apparent by describing in further detail exemplary embodiments thereof with reference to the accompanying drawings in which:

FIG. 1 shows the amino acid sequence of human interferon alpha 2b (Gull et al., SpringerPlus 2013, 2:264).

FIGS. 2A/2B show three identified isoforms of human INF alpha 2b that are formed as a result of conventional bacterial expression of interferon alpha 2b. (A) RP-HPLC separation of the isoforms, and their molecular weight differences; (B) shows their isoform structure.

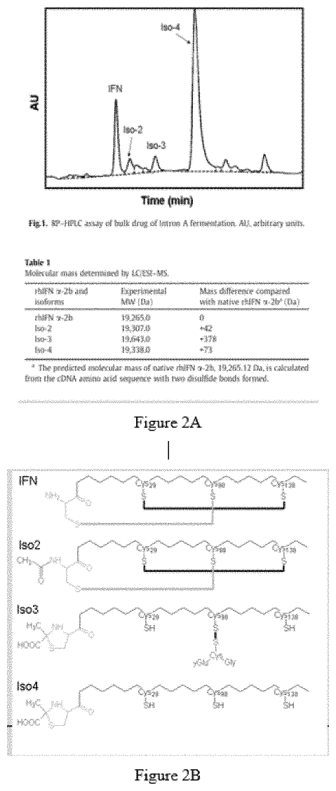

FIGS. 3A/3B show novel expression constructs for human INF alpha 2b fusion protein expression in accordance with the present invention. (A) amino acid sequence of the three constructs; (B) confirms expression of human INF alpha 2b protein by each construct and 10% SDS-PAGE (stained with Coomassie blue) purification of the tagged human INF α2b fusion protein isolated from E. coli extract.

FIG. 4 shows an AKTA chromatogram of first IMAC purification of thioredoxin-rhIFN α-2b fusion protein.

FIG. 5 shows an SDS-PAGE of material from the first IMAC purification with loading of the protein load, flow-through of the load, the post load wash, and elution fractions. Lanes contained approximately 5 μg of protein.

FIG. 6. RP-HPLC of material obtained from the first IMAC purification.

FIG. 7 shows a SDS-PAGE of thioredoxin-rhIFN α-2b following the enzymatic cleavage. Cleavage reaction was loaded in triplicate (Lanes 2-4). Lanes contained approximately 5 μg of protein.

FIG. 8 is a RP-HPLC of material after enzymatic cleavage with enterokinase, prior to the second IMAC purification.

FIG. 9 shows an AKTA chromatogram of the second IMAC purification of rhIFN α-2b protein.

FIG. 10 shows a SDS-PAGE with loading of the flow-through of the load, the post load wash, and elution fractions 6, 7 and 8 of the second IMAC purification step. Lanes were loaded with approximately 5 μg of protein.

FIG. 11 shows a RP-HPLC of load flow-through and wash material after the second IMAC purification.

FIG. 12 shows a RP-HPLC of elution fraction 7 from the second IMAC purification.

### DESCRIPTION OF EMBODIMENTS

### Definitions

As used herein, the terms “invention” or “present invention” are non-limiting terms and not intended to refer to any single aspect of the particular invention but encompass all possible aspects as described in the specification and the claims.

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in the art to which the subject matter herein belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the following definitions are supplied in order to facilitate the understanding of the present invention.

It will be understood that any component defined herein as being included may be explicitly excluded from the claimed invention by way of proviso or negative limitation. In addition, all ranges given herein include the end of the ranges and also any intermediate range points, whether explicitly stated or not.

As used herein, the articles “a” and “an” preceding an element or component are intended to be non-restrictive regarding the number of instances (i.e. occurrences) of the element or component. Therefore, “a” or “an” should be read to include one or at least one, and the singular word form of the element or component also includes the plural unless the number is obviously meant to be singular.

It will be further understood that the terms “comprises” and/or “comprising,” or “includes”, “including” and/or “having” and their inflections and conjugates denote when used in this specification, specify the presence of stated features, regions, integers, steps, operations, elements, and/or components, but do not preclude the presence or addition of one or more other features, regions, integers, steps, operations, elements, components, and/or groups thereof.

It will be understood that any component defined herein as being included in any described embodiment may be explicitly excluded from the claimed invention by way of proviso or negative limitation.

As used herein, the term “about” refers to variation in the numerical quantity. In one aspect, the term “about” means within 10% of the reported numerical value. In another aspect, the term “about” means within 5% of the reported numerical value. Yet, in another aspect, the term “about” means within 10, 9, 8, 7, 6, 5, 4, 3, 2, or 1% of the reported numerical value.

“About,” is equivalent to “approximately,” or “substantially” as used herein and inclusive of the stated value and means within an acceptable range of deviation for the particular value as determined by one of ordinary skill in the art, considering the measurement in question and the error associated with measurement of the particular quantity (i.e., the limitations of the measurement system). For example, “about,” “approximately,” or “substantially” can mean within one or more standard deviations, or within +30%, 20%, 10%, 5% of the stated value.

Should a range of values be recited, it is merely for convenience or brevity and includes all the possible sub-ranges as well as individual numerical values within and about the boundary of that range. Any numeric value, unless otherwise specified, includes also practical close values and integral values do not exclude fractional values. Sub-range values and practically close values should be considered as specifically disclosed values.

As will also be understood by one skilled in the art, all language such as “up to”, “at least”, “greater than”, “less than”, “more than”, “or more”, and the like, include the number recited and such terms refer to ranges that can be subsequently broken down into subranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

As used herein the term ‘may’ denotes an option or an effect which is either or not included and/or used and/or implemented and/or occurs, yet the option constitutes at least a part of some embodiments of the invention or consequence thereof, without limiting the scope of the invention.

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary.

As used herein, expressions such as “at least one of,” when preceding a list of elements, modify the entire list of elements and do not modify the individual elements of the list.

“Combination or combining” for the purposes of this invention means any method of putting two or more materials together. Such methods include, but are not limited to, mixing, blending, commingling, concocting, homogenizing, incorporating, intermingling, fusing, joining, shuffling, stirring, coalescing, integrating, confounding, joining, uniting, or the like.

The term “pharmaceutically acceptable” means that the compound or combination of compounds is compatible with the remaining ingredients of the formulation for pharmaceutical use, and that it is generally safe for administering to humans according to established governmental standards.

The term “pharmaceutically acceptable carrier” includes, but is not limited to a diluent, adjuvant, excipient or vehicle with which an active agent is administered. This encompasses standard pharmaceutical carriers such as a phosphate buffered saline solution, and water.

As used herein, the term “excipient” refers to any inert and pharmaceutically acceptable material that has substantially no biological activity, and makes up a substantial part of the composition so as to aid the administration of an active agent to a subject.

As used herein the term “pegylated interferon” means covalent conjugates of one or more polyethylene glycol (PEG) molecules and one or more interferon molecules. Conjugates are preferred between a single PEG molecule and a single interferon molecule. Methods of conjugation of PEG and interferon molecules may be performed by any conjugation reaction known to those skilled in the art, for example as described in U.S. Pat. Nos. 5,612,460, 5,711,944 and 5,951,974 (the disclosures of which are herein incorporated by reference in their entirety).

As used herein, the term “force control agent” refers to an excipient utilized to control interparticle cohesive forces between particles. Force control agents are typically hydrophobic and include, but are not limited to, magnesium stearate, leucine, and long-chain saturated phospholipids (e.g., dipalmitoylphosphatidylcholine).

As used herein, the term “treating” refers to providing an appropriate dose of a therapeutic agent to a subject suffering from an ailment. An “acute” treatment (and/or an acute effect of such treatment) has an abrupt onset and short duration of action (e.g., less than 1 day), where as a “chronic” treatment lasts over a longer period of time (e.g., greater than three months).

“Alleviate” as used herein, is meant to include complete elimination as well as any clinically or quantitatively measurable reduction in the subject's symptoms and/or discomfort.

As used herein, “subject”/“patient” refers to a mammal that may benefit from the administration of the recombinant isoform free INFα 2b formulations of this invention. Examples of subjects include humans. In one specific aspect, a subject is a human.

An “effective amount” or a “therapeutically effective amount” refers to a non-toxic amount effective to treat to which this phrase refers, this can be a disease, disorder, and/or condition, or to bring about a recited effect. Determination of a therapeutically effective amount is well within the capacity of persons skilled in the art. The term “effective amount” is intended to include an amount of a compound described herein, or an amount of a combination of compounds described herein for providing the recited effect to a subject. Thus, an “effective amount” generally means an amount that provides the desired effect. While the achievement of therapeutic effects may be measured by a physician or other qualified medical personnel using evaluations known in the art, it is recognized that individual variation and response to treatments may make the achievement of therapeutic effects a somewhat subjective decision.

As used herein, the terms “administration,” and “administering” refer to the manner in which an active agent is presented to a subject. Administration can be accomplished by various art-known routes such as inhalation.

The term “pulmonary administration” represents any method of administration in which an active agent can be administered through the pulmonary route by inhaling an aerosolized liquid or powder form (nasally or orally). Such aerosolized liquid or powder forms are traditionally intended to substantially release and or deliver the active agent to the epithelium of the lungs. In certain embodiments, the active agent is in powder form.

The term “nasal administration” represents administration in which the drug/medicament is insufflated through the nose. It can be either inhalation of an aerosolized liquid or powder form or topical administration. Nasal administration is primarily for local effects.

As used herein, the phrase “aerosol delivery” means administration of a fine spray, mist, or colloidal suspension in the air. The term “aerosolization” refers to a process whereby a liquid formulation is converted to an aerosol. Representative devices for aerosolization include a jet nebulizer, an ultrasonic nebulizer, a metered dose inhaler, and an aerosolization device based on forced passage through a nozzle. The resulting compositions are referred to herein as “aerosol” compositions.

The phrase “suitable for pulmonary delivery” means that a protein included in the composition remains biologically active following pulmonary delivery.

As used herein, the terms “nominal dose”, “nominal load”, “total load,” and “ND” refer to the mass of drug that is administered in a single dose. That is, the total amount of formulation packaged or partitioned for administration to a subject in a single dose. For example, the nominal load is the total amount of powder formulation of the invention that is enclosed in a capsule for use with an inhaler.

The term “fine particle fraction” (% FPF(ED)) refers to the mass of active agent having an aerodynamic diameter below 5 μm expressed as a percentage of the emitted dose. The FPF is often used to evaluate the efficiency of aerosol deagglomeration.

The term “mass median aerodynamic diameter” (MMAD) refers to the mass median aerodynamic diameter of airborne particles at which 50% of particles by mass are larger and 50% are smaller.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### INTERFERON/rhIFN α2b

Interferons are a group of glycoprotein cytokines that block replication of DNA and RNA viruses by activating antiviral immunological reactions to unite against the virus. The IFN-α proteins are produced mainly by plasmacytoid dendritic cells (pDCs).

Human interferons are classified into three major types based on the type of receptor through which they signal. Interferon type I (IFN) binds to a specific cell surface receptor complex known as the IFN-α/β receptor (IFNAR) on target cells, produced by fibroblasts and monocytes when the body recognizes a virus that has invaded it, that consists of IFNAR1 and IFNAR2 chains, binding leads to expression of proteins that will prevent the virus from producing and replicating its RNA and DNA. The type I interferons (TFNI) present in humans are IFN-α, IFN-β, IFN-ε, IFN-κ and IFN-ω. Interferon type II (IFN-γ), binds to the IFNGR receptor, which consists of IFNGR1 and IFNGR2 chains and is activated by Interleukin-12 and released by cytotoxic T cells and T helper cells.

IFN-α and IFN-β are secreted by many cell types including lymphocytes (NK cells, B-cells and T-cells), macrophages, fibroblasts, endothelial cells, osteoblasts and others. They stimulate both macrophages and NK cells to elicit an anti-viral response, involving IRF3/IRF7 antiviral pathways, and are also active against tumors.

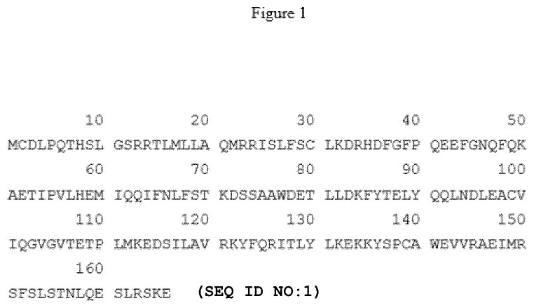

Human IFN-α2b is a 19.3 kDa protein containing 165 amino acid residues (FIG. 1) (Bekisz, et al., Growth Factors, December 2004. Vol 22(4), pp 243-251). In aspects, the invention provides a recombinant (authentic) isoform free human interferon α 2b protein (rhIFN α2b), compositions and formulations comprising the rhIFN α2b. The rhIFN α2b of the invention is synthesized in an E. coli host using a novel construct that does not express undesired isoforms. As a result the eluted human alpha interferon alpha 2b does not require complex purification steps to remove undesired isoforms. The rhIFN α2b of the invention is surprisingly stable as a composition formulated for therapeutic administration such that it has a long shelf/storage life at different temperature conditions. Stability also helps to lessen off target local and systemic effects.

The rhIFN α2b of the invention is unconjugated, however, it can be pegylated or fused with human serum albumin (HSA).

The rhIFN α2b of the invention can be provided as a composition in sterile buffer (such as a sodium phosphate buffer) and/or sterile water at a pH of about 7.5+/−0.3. The sterile composition can be lyophilized and frozen and further reconstituted. Desired concentrations of the rhIFN α2b are made by admixing the rhIFN α2b in the sterile liquid as the rhIFN α2b is soluble.

### Viruses/Respiratory Viruses

Formulations of recombinant isoform free human IFN α-2b of the invention are suitable for the treatment and/or prevention of viral respiratory infections and/or for minimizing one or more symptoms of a respiratory infection. In aspects of the invention, examples of viruses causing respiratory infection that may be targeted with formulations as described herein are: coronavirus (alpha, beta, gamma and delta) such as human coronaviruses 229E (alpha coronavirus), NL63 (alpha coronavirus), OC43 (beta coronavirus), HKU1 (beta coronavirus), MERS-CoV (beta coronavirus causing Middle East respiratory syndrome), SARS-CoV (beta coronavirus causing severe acute respiratory syndrome), and SARS-CoV-2 (new coronavirus causing COVID-19); influenza viruses; parainfluenza viruses; adenoviruses; rhinoviruses; and meta-pneumoviruses.

In one aspect of the invention, formulations of recombinant isoform free IFN-α 2b is for the treatment and/or prevention of COVID-19.

With the surprising stability of the formulations of the isoform free rhIFN α-2b of the invention, the formulation is safe and effective for intravenous, intramuscular, intrathecal, parenteral, intrathecal and intravaginal administration. Administration can be of any manner where the formulations of the invention would benefit a patient. This would also include patients at risk or with infection by Flaviviruses (Dengue, Zika), West Nile virus, tick borne encephalitis virus, Filoviruses such as Ebola, Bunyaviruses such as Hantavirus, and Hepdna viruses such as Hepatitis A, B & C as understood by one of skill in the art to help alleviate at least one symptom of the aforementioned viruses.

### Patient/Subject

In an embodiment, the recombinant isoform free hIFN α-2b of the invention is suitable for stable formulation for administration to a patient/subject for treatment or reduction of a respiratory viral infection. The patient/subject may be at higher risk for respiratory infection due to the presence of one or more risk factors/pre-existing conditions such as age, weight, high blood pressure, chronic kidney disease, HIV infection, immune disorder, use of auto-immune medication for underlying disorder, diabetes, pre-existing pulmonary disease (e.g. COPD), heart conditions and asthma.

The patient/subject may be at higher risk for infection as a result of working in an environment with potential high possibility of infection, for example first responders (ambulance and fire department personnel), hospital workers, transportation workers, and the like.

The patient/subject may be anyone that desires taking preventative measures in order to minimize risk of respiratory infection, in aspects, in a pandemic.

### Formulations—Pulmonary

The present invention provides stable formulations comprising a pure recombinant isoform free human IFN α-2b for administration to the respiratory system for the treatment of viral pulmonary infection such as COVID-19. The recombinant isoform free human IFN α-2b formulations of the invention are formulated for aerosol administration to the lungs of a patient or formulated for nasal administration. The physical and chemical stability of the recombinant isoform free IFN α-2b formulation is maintained under a variety of less than ideal or adverse conditions such that when the patient eventually administers the formulation, the integrity and percentage of the active agent actually delivered to the patient is maintained as compared to a heterogeneous IFN formulation that is stored under “ideal” storage conditions.

Formulations of the invention for administration comprise pure recombinant isoform free IFN α-2b, stabilizing agent(s), buffer(s) and pharmaceutically acceptable carrier(s)/excipient(s). The stabilizing agent stabilizes the rhIFN α-2b within the formulation to prevent/minimize degradation thereof over an extended period of storage under various temperatures. The carriers and excipients are selected on the basis of the format of the formulation, whether for oral or nasal administration, for example, whether a liquid aerosol or a solid dry powder aerosol, nebulized droplets (i.e. mist), spray or gel.

Surprisingly, the recombinant isoform free human IFN α-2b formulations are stable at freezing, room and refrigerated temperatures over extended time. Formulations comprise one or more stabilizing agents for stabilization of the recombinant isoform free human IFN α-2b extending biological activity and shelf life of the formulations. In an aspect the stabilizing agent functions for example as an antioxidant that can prevent oxidation or dimerization of cysteine in the protein. Suitable anti-oxidant stabilizing agents are water soluble and selected from the group consisting of methionine, cysteine, vitamin C (D-ascorbic acid/sodium ascorbate), glutathione, lipoic acid, uric acid and combinations thereof. In aspects the stabilizing agent is lipoic acid. In aspects the stabilizing agent is methionine. The amount of stabilizing agent provided in formulations may comprise about 0.01 to about 5.0 mg/ml. In aspects up to about 0.5 mg/ml, up to about 1.0 mg/ml, up to about 1.5 mg/ml, up to about 2.0 mg/ml, up to about 2.5 mg/ml, up to about 3.0 mg/ml, up to about 3.5 mg/ml, up to about 4.0 mg/ml, up to about 4.5 mg/ml or up to about 5.0 mg/ml. Stabilized formulations of the invention maintain a clear and colorless appearance free of particulate matter and free of bacterial endotoxin for extended periods of time at temperatures of about −20° C.+/−5° C.; about 2° C.+/−3° C. to about 8° C. +/−3° C.; and about 25° C.+/−2° C. and 40° C.+/−2° C. while maintaining a desired pH of about 7.5+/−0.3 and desired osmolality.

Pharmaceutically acceptable carriers and excipients for formulations may include buffering agents such as sodium phosphate, sodium acetate, sodium carbonate, citrate, glycylglycine, histidine, HEPES, arginine, TRIS, glycine and sodium citrate or mixtures thereof. More specifically, suitable pharmaceutically acceptable excipients for use in the inhalation formulations of the invention in varying embodiments may be selected for example from one or more of acetic acid (glacial), acetyltributyl citrate, albumin, ascorbic acid, Cetostearyl Alcohol, Calcium Phosphate, Dibasic Dihydrate/anhydrous, Citric Acid, Copovidone, Dextrates, Dextrin Dextrose, Ethyl Oleate, Fructose, Fumaric Acid, Gelatin, Glucose, Liquid Glycerin, Glyceryl Behenate, Glyceryl Monooleate, Glyceryl Monostearate, Glyceryl Palmitostearate, Hydrochloric Acid, Hydroxyethyl Cellulose, Lecithin, Leucine, Linoleic Acid, Magnesium Carbonate, Maltitol, Maltodextrin, Maltol, Maltose, Mannitol, Medium-chain Triglycerides, Methylcellulose, Octyldodecanol, Oleic Acid, Palmitic Acid, Hydroxyethylmethyl Cellulose, Hydroxypropyl Cellulose, Hydroxypropyl Cellulose, Low-substituted, Hydroxypropyl Starch, Hypromellose, Hypromellose Acetate, Succinate, Dimethyl Sulfoxide, Disodium Edetate, Edetic Acid, Erythorbic Acid, Erythritol, Ethyl Acetate, Lactic Acid, Lactitol, Lactose, Anhydrous, Lactose, Monohydrate, Lactose, Spray-Dried, Ethyl Lactate, Ethyl Maltol, Hypromellose Phthalate, Ethylcellulose, Saccharin, Saccharin Sodium, potassium alginate, potassium citrate, potassium chloride, Simethicone, Thymol, Sodium Acetate, Titanium Dioxide, Sodium Alginate, Sodium Ascorbate, Trehalose, Sodium Benzoate, Triacetin,Sodium Bicarbonate, Tributyl Citrate, Sodium Borate, Sodium Chloride,Triethyl Citrate,Sodium Citrate, Dihydrate Sodium, Cyclamate Sodium Hyaluronate, Water, Sodium Hydroxide, Sodium Lactate, Sodium Lauryl Sulfate, Sodium Metabisulfite, Sodium Phosphate, Dibasic Sodium Phosphate, Monobasic Sodium Propionate, Sodium Starch Glycolate, Sodium Stearyl Fumarate, Xylitol, Sodium Sulfite, Sorbic Acid, Zinc Acetate, Sorbitan Esters (Sorbitan Fatty Acid Esters), Zinc Stearate, Sorbitol, Soybean Oil, Starch, Pregelatinized Starch, Stearic Acid, Stearyl Alcohol, Sucralose, Sucrose Sugar, Compressible Sugar, Sulfobutylether, 13-Cyclodextrin, Sulfuric Acid, and Tartaric Acid.

The recombinant isoform free human IFN α-2b formulations of the invention may further comprise one or more additional antiviral agent(s).

Formulations comprising the rhIFN α-2b of the invention can be made in a variety of concentrations and as used with a device, can provide a volume dose(s) with a desired therapeutic amount of recombinant isoform free human IFN α-2b per dose. For example, therapeutic formulations of the present invention for aerosol/nebulized droplets/spray administration may comprise about 0.01 mg/ml to about 5 mg/ml recombinant isoform free human IFN α-2b, in aspects 0.01 mg/ml to about 1.0 mg/ml. The purity of the recombinant isoform free IFN α-2b, combined with stability allows lower dosing and still maintain effective anti-viral activity. Lower dosing at more frequent intervals further allows for more frequent dosing to maintain a level of more consistent anti-viral activity without negative systemic and/or local effects. Doses of 0.01 mg/ml may be administered more frequently to a patient. Dosages can be expressed in MIU, symbol for one million international units (IU). For example, the formulations for aerosol inhalation may contain about 5 MIU rhIFN α-2b via inhalation and be suitable for twice a day administration for up to one week or up to two weeks.

An aerosol formulation may comprise up to about 2.5 mg/ml rhIFN α-2b, up to about 5.0 mg/ml sodium phosphate dibasic, up to about 5.0 mg/ml sodium phosphate monobasic, up to about 1.0 mg/ml EDTA, up to about 1.0 mg/ml polysorbate 80 and up to about 5.0 mg/ml stabilizing agent comprising methionine.

The formulations of the invention are suitable for delivery to patients with respiratory viral infections or at risk of developing respiratory viral infections that exhibit highly compromised lungs, patients lacking the capacity to strongly inhale (young and old patients, infirm patients) and patients with asthma or other breathing difficulties. Patients suffering from a viral respiratory infection may simply lack the ability to sufficiently inhale. The formulations of the invention by virtue of being isotypically pure and stable are effective, in aspects more effective, to reduce and/or prevent viral replication and thus reduce and/or ameliorate infection while not exerting undesired local and/or systemic effects.

Formulations of the invention as a clinical aerosol can be used with a device for example selected from a jet or ultrasonic nebulizer; metered dose inhaler (MDI); and dry powder inhaler. Each functions to generate an aerosol cloud of the rhIFN α-2b isoform free formulation that contains the highest possible fraction of particles (liquid or solid) in a desired size range and each are suitable for use with the present invention. The aerosol stable formulations deposit in the airways by gravitational sedimentation, inertial impaction, and diffusion.

Formulations are delivered by administration, in a single breath, to a patient's respiratory tract the recombinant isoform free human IFN α-2b in the container of the device. Expressed in MIU, at least 1 MIU/ml, at least 1.5 MIU/ml, at least 2.0 MIU/ml, at least 2.5 MIU/ml, at least 3.0 MIU/ml, at least 3.5 MIU/ml, at least 4.0 MIU/ml, at least 4.5 MIU/ml or at least 5.0 MIU/ml isoform free rhIFN α-2b stable formulation may be used in conjunction with a delivery device.

In aspects, formulation solutions of about 10 μg to about 100 μg of recombinant isoform free IFN α-2b are made from which suitable doses are determined. In one non-limiting example, a solution of 10 μg of recombinant isoform free IFN α-2b provides doses of 0.01 mg/ml. For example a 2.5 MIU/ml solution of recombinant isoform free IFN α-2b is incorporated within a nebulizer for administration of the formulation. A sterile single dose container for use with a nebulizer may contain 3 mls of 2.5 MIU/ml recombinant isoform free IFN α-2b. A patient may inhale 2 mls to deliver 5.0 MIU of the recombinant isoform free IFN α-2b in one dose. Dosage may be repeated twice daily for 14 days. Alternatively, dosage may be decreased for repeating four times daily for 14 days.

As formulated for use as a dry powder aerosol for inhalers, dry powder nanoparticle aerosol may comprise a hygroscopic excipient. The formulation is provided as particles of dry powder that may comprises for example, buffer salts, dextran, polysaccharides, lactose, trehalose, cyclodextrins, proteins, peptides, polypeptides, fatty acids, fatty acid esters, inorganic compounds, and phosphates. Particles are up to about 5 microns, up to about 4.5 microns, up to about 4.0 microns, up to about 3.5 microns, up to about 3.0 microns, up to about 2.5 microns, up to about 2.0 microns, up to about 1.5 microns, up to about 1.0 microns or up to about 0.5 microns. A “dry powder” or “powder” as used herein with regard to the particles and the formulations of the invention means that the moisture content of the mass of particles is generally below about 10% by weight of water, more preferably below about 5% by weight of water and preferably less that about 3% by weight of water.

The pulmonary formulations of recombinant isoform free IFN α-2b may be provided as spray dried dry powder particles having physical characteristics characterized by a fine particle fraction (FPF) favoring target lung deposition to optimize release and bioavailability. As used herein, the term “fine particle fraction” of a collection of particles refers to the fraction by weight, typically expressed as weight percent, of the total powder which is present as particles of aerodynamic diameter less than 3.3 μm. FPF of the formulations of the invention is at least about 20% and up to about 90%. The particles can be fabricated with a rough surface texture to reduce particle agglomeration and improve flowability of the powder. The spray-dried particles have improved aerosolization properties. The spray-dried particle can be fabricated with features which enhance aerosolization via dry powder inhaler devices, and lead to lower deposition in the mouth, throat and inhaler device.

The pulmonary formulations of recombinant isoform free IFN α-2b aerodynamically light particles may have a desired “mass median aerodynamic diameter” (MMAD), between about 1 μm and about 5 μm or any subrange encompassed between.

The invention also includes administering the dry powder particles of recombinant isoform free IFN-α 2b from a device having, holding, containing, storing or enclosing a mass of the particles, to a subject's respiratory tract. For example, particles are delivered from an inhalation device such as a dry powder inhaler (DPI) or metered-dose-inhalers (MDI). At least 80% of the mass of the biocompatible particles held in the inhaler device is delivered to a subject's respiratory system in a single, breath-activated step. The device may hold the formulation within a capsule, blister, film covered container well, chamber and other suitable means of storing a powder in an inhalation device known to those skilled in the art. In one embodiment of the invention, the article or powder enclosed or stored in the receptacle have a mass of at least about 0.1 milligram to at least about 20 milligrams. In one embodiment, the powder enclosed or stored in the receptacle is present in an amount of at least 0.1, 0.3, 0.6, 0.9, 1, 3, 5, 7, 10, 13, 15, 17, 20, 23, 25, 27, or 30 milligrams.

In aspects the device is a dry powder inhaler such as for example disclosed is U.S. Pat. Nos. 4,995,385 and 4,069,819, the SPINHALERO. (Fisons, Loughborough, U.K.), ROTAHALER®. (Glaxo-Wellcome, Research Triangle Technology Park, North Carolina), FLOWCAPS®. (Hovione, Loures, Portugal), INHALATOR®. (Boehringer-Ingelheim, Germany), and the AEROLIZER®. (Novartis, Switzerland), the Diskhaler (Glaxo-Wellcome, RTP, NC), ULTRAVENT™ (Mallinckrodt, Inc.), the ACORN II™ (Marquest Medical Products), VENTOLIN™ metered dose inhaler (Glaxo Inc.) and the SPINHALER™ powder inhaler (Fisons Corp.) MISTYNEB™ (Allegiance), and AEROECLIPSE™ (Trudell Medical International, Canada), Aerogen™ pulmonary delivery technology, and others known to those skilled in the art.

The AERx pulmonary drug delivery system (U.S. Pat. Nos. 5,660,166; 5,718,222; 5,823,178; 5,497,763; and 5,544,646, the entirety of each of these patents is incorporated herein by reference) is also suitable for use with the compositions of the invention for treatment and/or prophylaxis of COVID-19.

Formulations for use with a metered-dose inhaler device may generally comprise a finely divided powder containing the protein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

The invention also provides a kit comprising an inhalation device comprising one or more doses of formulated stable recombinant isoform free human IFN α-2b, suitable for inhalation to provide up to about 5.0 MIU per inhalation. The kit may also contain instructions for use. The kit can be disposable after one use or configured to provide a number of doses before disposal or refilling thereof. The recombinant isoform free IFN α-2b may be formulated specific to the type of device employed.

The stable formulations for inhalation are particularly useful for the treatment of hospitalized patients using a nebulizer. The recombinant isoform free IFN α-2b is dissolved/reconstituted in sterile water at a concentration of about 0.01 mg/ml to about 1 mg/ml of solution. The formulation may also include a buffer and a carbohydrate such as a simple sugar (e.g., for protein stabilization and regulation of osmotic pressure). Suitable carbohydrates include monosaccharides, oligosaccharides or polysaccharides. Examples include but are not limited to glucose, maltose, maltotriose, maltotetraose, sucrose and trehalose. The nebulizer formulation may also contain a surfactant, to reduce or prevent surface induced aggregation of the protein caused by atomization of the solution in forming the aerosol. The aerosol will create a very large surface to air ratio for the formulation (3×10⁶ m²/m³ for a 2 μm droplet), the surfactants also protect the protein from denaturation at the interface and may include polysorbates, such as polysorbate 20, poloxamers, such as poloxamer 188, and polyethyleneoxide glycol (PEG).

In one aspect, a goal of aerosol delivery to significantly increase the targeted delivery of IFN α-2b isoform free composition to the deep lung in humans. SARS-COV-2 can cause significant lung tissue damage resulting in geometric changes in the lung periphery that can minimize the deposition of inhaled particles. The recombinant isoform free IFN α-2b formulations can advantageously be aerosolized and thus delivered directly to the site of disease (the lung periphery) and thus more effective at reducing infection.

The stable formulations of the invention can be used/administered to a patient/subject before, during, and/or after other treatments for a respiratory virus. In one aspect, formulations of the invention can comprise one or more other anti-viral agent such as but not limited to: arbidiol, remdesivir, lopinavir/ritonavir (Kaletra™), oseltamivir (Tamiflu™), favipiravir (Avigan™), Favipiravir (Gilenya™), Methylprednisolone, Chloroquine phosphate, Hydroxychloroquine sulfate, Hydroxychloroquine and azithromycin, Leronlimab, Ivermectin, Sarilumab and combinations thereof.

### Nasal Formulations

In accordance with a further aspect of the invention are nasal formulations of recombinant isoform free IFN α-2b composition for nasal delivery (insufflation). The nose provides a portal for the entry of viruses and passage to mucous membranes in the throat before moving down the respiratory tract. Therefore nasal formulations comprising a single isoform rIFN α-2b composition are useful to target an early point of viral entry to prevent the virus from infecting mucosal cells and spreading. Nasal delivery provides a localized administration and an early immune response. Nasal delivery has minimal systemic effects. Nasal formulations can be used to prevent viral infections such as SARS-COV2 infection.

Nasal administration is suitable for administering a limited volume of the formulation into the nasal cavity. There is about 20 mL capacity in the adult human nasal cavity. The major part of the approximately 150 cm² surface in the human nasal cavity is covered by respiratory epithelium, across which systemic drug absorption can be achieved. The olfactory epithelium is situated in the upper posterior part and covers approximately 10 cm² of the human nasal cavity.

Nasal formulations may comprise agents to improve bioavailability of the IFN in the nasal mucosa to improve nasal residence time and/or to enhance nasal absorption, such agents may include nasal enzyme inhibitors (e.g. peptidases, proteases, tripsin, aprotinin, borovaline, amastastin, bestatin and broleucin inhibitors) and permeation enhancers (inhibit nasal enzyme activity, reduce mucus viscosity or elasticity, decrease mucociliary clearance, and open tight junctions) that are compatible with excipients used in the formulation. Permeation enhances may include but not be limited to bile salts and derivatives, surfactants, chelating agents, fatty acids, bioadhesive materials and liquids. Alternatively, agents to improve bioavailability can be formulated separately for initial use prior to the administration of the recombinant isoform free human IFN α-2b nasal formulation. This may be provided as a kit with instructions for use.

Nasal formulations of single isoform rhIFN-α2b can be in the form of nasal gels, nasal drops, nasal sprays, nasal powders, ointment, cream, liposomes and microspheres. Gels, ointments and creams reduce any possible drip due to high viscosity, reduce any impact of taste and reduce any anterior leakage. Irritation is reduced when using emollient excipients. Nasal drops are convenient but not provide dose precision. The recombinant isoform free hIFN α-2b formulation can be carried within microspheres (for example using polymers such as dextrin, chitosan, biodegradable starch), nanoparticles and liposomes for increased adsorption efficacy and stability and decreased undesired effects. This may result in increased mucoadhesion to increase retention time.

Microspheres provide prolonged contact with the nasal mucosa and swell upon contact thereto. Microsphere particle size can be up to about 50 microns.

Nasal sprays (solution and suspension formulations) are used in conjunction with metered dose pumps and actuators of varying construction to provide a desired particle size and morphology and viscosity of formulation. Nasal sprays can provide exact dosing.

Nasal stable formulations of rhIFN α-2b can be used alone or in combination with other nasal actives. Administration is as an aerosol or provided as a solution for use with a nebulizer.

The descriptions of the various embodiments and/or examples of the present invention have been presented for purposes of illustration but are not intended to be exhaustive or limited to the embodiments and/or examples disclosed. Many modifications and variations will be apparent to those of ordinary skill in the art without departing from the scope and spirit of the described embodiments. The terminology used herein was chosen to best explain the principles of the embodiments, the practical application, or to enable further understanding of the embodiments disclosed herein.

The present invention will now be described in more detail with the following non-limiting Examples:

### EXAMPLES

These examples are not intended to limit the scope of the invention nor intended to represent that the experiments below are all or the only experiments performed.

### Example One

### Creation of INFα2b Expression Clones

The E. coli-optimized nucleic acid sequence encoding INFα2b was inserted into each of three plasmids driven by one of three inducible promoters: a Rhamnose promoter, a T5 promoter or a T7 promoter. Each of the three plasmids express a sequence encoding INFα2b, with no tags, intracellularly. The inducible promoters drive expression of the following human INFα2b sequence:

(SEQ ID NO: 1)
MCDLPQTHSLGSRRTLMLLAQMRRISLFSCLKDRH
DFGFPQEEFGNQFQKAETIPVLHEMIQQIFNLFST
KDSSAAWDETLLDKFYTELYQQLNDLEACVIQGVG
VTETPLMKEDSILAVRKYFQRITLYLKEKKYSPCA
WEVVRAEIMRSFSLSTNLQESLRSKE.

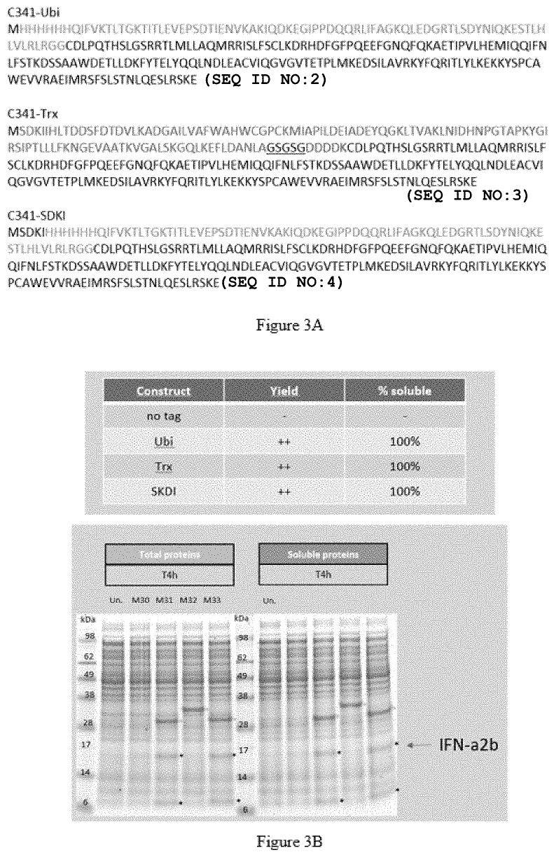

Six plasmids were constructed to incorporate one of the three promoters (Rhamnose promoter, a T5 promoter or a T7 promoter) to drive two gene sequences to be expressed that possess a fusion moiety and an affinity tag. These vectors express intracellularly (FIG. 3A):

HIS affinity tag-ubiquitin-
(SEQ ID NO: 14)
CDLPQTHSLGSRRTLMLLAQMRRISLFSCLKDRHD
FGFPQEEFGNQFQKAETIPVLHEMIQQIFNLFSTK
DSSAAWDETLLDKFYTELYQQLNDLEACVIQGVGV
TETPLMKEDSILAVRKYFQRITLYLKEKKYSPCAW
EVVRAEIMRSFSLSTNLQESLRSKE
HIS-Patch thioredoxin-DDDDK-
(SEQ ID NO: 15)
CDLPQTHSLGSRRTLMLLAQMRRISLFSCLKDRHD
FGFPQEEFGNQFQKAETIPVLHEMIQQIFNLFSTK
DSSAAWDETLLDKFYTELYQQLNDLEACVIQGVGV
TETPLMKEDSILAVRKYFQRITLYLKEKKYSPCAW
EVVRAEIMRSFSLSTNLQESLRSKE

Three plasmids were constructed to incorporate one of the three promoters (Rhamnose promoter, a T5 promoter or a T7 promoter) to drive expression of the gene sequence for INFα2b using one signal sequence (ompC). These vectors express in the periplasmic space.

OmpC-
(SEQ ID NO: 14)
CDLPQTHSLGSRRTLMLLAQMRRISLFSCLKDRHD
FGFPQEEFGNQFQKAETIPVLHEMIQQIFNLFSTK
DSSAAWDETLLDKFYTELYQQLNDLEACVIQGVGV
TETPLMKEDSILAVRKYFQRITLYLKEKKYSPCAW
EVVRAEIMRSFSLSTNLQESLRSKE

Gene expression analysis of the different expression plasmids in various E. coli strains was examined. E. coli strains were transformed according to the specific plasmid:

The Rha promoter vectors into a BL21 Rha- and a W3110 Rha-host;

The T5 promoter vectors into a BL21 and a W3110 host;

The T7 promoter vector into a BL21 T7-specific host; and

Rha and T5 promoter vectors specific for cytoplasmic expression transformed into an E. coli shuffle host.

Different vector/host clones analysis for expression:

Intracellular expression, no tags

| promoter | W3110 rha- | BL21 rha- | BL21 T7 | Shuffle |
|---|---|---|---|---|
| T5 | X | X | | X |
| T7 | | | X | |
| Rhamnose | X | X | | X |

Periplasmic expression, with OmpC

| promoter | W3110 rha- | BL21 rha- | BL21 T7 | Shuffle |
|---|---|---|---|---|
| T5 | X | X | | |
| T7 | | | X | |
| Rhamnose | X | X | | |

Intracellular expression, with Ubi tag

| promoter | W3110 rha- | BL21 rha- | BL21 T7 | Shuffle |
|---|---|---|---|---|
| T5 | X | X | | X |
| T7 | | | X | |
| Rhamnose | X | X | | X |

Intracellular expression, with HIS-patch thioredoxin-Ent tag

| promoter | W3110 rha- | BL21 rha- | BL21 T7 | Shuffle |
|---|---|---|---|---|
| T5 | X | X | | X |
| T7 | | | X | |
| Rhamnose | X | X | | X |

All clones were grown and induced in animal-free LB medium at incubation temperatures between 20° C. to 30° C. (at 30° C. some inclusion body formation may be observed).

Samples taken post induction, and analyzed by 10% SDS-PAGE for the level of recombinant protein expression shown in FIG. 3B. The samples first normalized by optical density (an OD=3 provides excellent standardization and resolution of protein bands) before loading onto SDS-PAGE.

### Example Two

### Expression of Thioredoxin/Recombinant Human Interferon α-2b (rhIFN α-2b) Fusion in Recombinant Escherichia coli Strain W3110 and Purification of Recombinant Human Interferon α-2b (rhIFN α-2b)

Purified rhIFN α-2b was obtained from a thioredoxin fusion protein product within E. coli lysate. The purified product was free from contaminating isoforms. The purified product demonstrated a purity of 100% relative to isoforms and other product related impurities by RP-HPLC.

Recombinant Escherichia coli strain W3110 was transfected with a construct (example 1) to intracellularly express a fusion protein of thioredoxin and recombinant human interferon α-2b (rhIFN α-2b) within the soluble fraction of cell lysate. The thioredoxin protein was inserted between the N-terminal methionine and cysteine residues of rhIFN α-2b to prevent the post-translational formation of isoforms, the expression under control of an IPTG-inducible T5 promoter.

### Culture

Cultured E. coli transfected with the thioredoxin-rhIFN α-2b fusion construct was induced to synthesize recombinant human interferon α-2b (rhIFN α-2b). Cultures began from colonies on streak plates or cryogenically frozen cells that are expanded in a shake flask. An aliquot of cells was added to a shake flask containing APF-LB (animal-product free Luria Bertani) medium supplemented with kanamycin and placed within a rotary shaker. The culture incubated at 30° C. with continuous shaking.

After cell expansion within a shake flask, the culture was inoculated into a bioreactor operated in fed-batch mode. The medium for the batch phase is a chemically-defined rich medium supplemented with glucose or glycerol and kanamycin. Production batch fermentation was conducted by inoculating batch medium with cells obtained from the shake flask culture. Temperature maintained at 30° C., pH maintained at 6.8 with the addition of 28-30% (w/w) NH₄OH or 33% (w/w) H₃PO₄, as needed, and dissolved oxygen concentration maintained at ≥30% through the use of agitation, aeration, or supplemental oxygen. Observation of a spike in dissolved oxygen, indicating depletion of the initial amount of carbon source indicates the end of the batch phase.

The fed-batch phase increases the biomass level beyond that achieved in the batch phase. After the increase in pO2 signaling the depletion of batch phase carbon source, a linearly increasing feed is initiated. The feed rate was chosen to match a target growth rate of 0.13/h during the fed-batch phase with feeding occurring over a 13-hour period. The feed consists of glycerol, yeast extract, and soytone (a hydrolysate of soy protein as a source of amino acids, any hydrolysate could serve the same purpose, such as a hydrolysate of wheat protein, casein, whey, almond, coconut, meat, fish. Non-animal derived sources are preferred). Throughout fed-batch phase, pH, temperature, and dissolved oxygen control remain the same as within the batch phase.

The last phase of the fermentation process is the induction phase. The feed from the fed-batch phase changes from linearly increasing to a constant level. Dissolved oxygen and pH controls are maintained the same as the batch and fed-batch phase. Temperature was lowered from 30° C. to 26° C. Induction began with the addition of IPTG, which induces expression from the T5 promoter system. Titers from cultures are anticipated to yield 0.3-0.6 g/L of rhIFN α-2b.

At the end of the induction phase, culture broth was collected and cells separated from supernatant via a combination of centrifugation and filtration. Supernatant is discarded as the product is intracellularly expressed. Cells are resuspended in a lysis buffer into which the product is extracted. Intracellular product is released via lysing cells. Cells can be lysed using osmotic shock, enzymatic lysis, or mechanical lysis means.

### Purification

A purified recombinant human interferon α-2b is obtained from a thioredoxin fusion protein product within Escherichia coli lysate. Purification consisted of immobilized metal affinity chromatography followed by enzymatic cleavage to separate the thioredoxin fusion protein from recombinant human interferon α-2b. The purified product was free from contaminating isoforms. Additional ion exchange chromatography can be employed to increase product purity. Purified rhIFN α-2b does not contain an N-terminal methionine or other N-terminal modifications, as the methionine residue is removed during cleavage from the N-terminal thioredoxin fusion protein.

An overview of the purification scheme for rhIFN α-2b purification from E. coil lysate is shown in the following: cell lysate; then first immobilized metal affinity chromatography purification (purification of thioredoxin-rhIFN α-2b fused protein); then second immobilized metal affinity chromatography purification (purification of thioredoxin-rhIFN α-2b fused protein); then enterokinase enzymatic cleavage (enzymatic separation of thioredoxin from rhIFN α-2b); then second immobilized metal affinity chromatography purification (purification of rhIFN α-2b from thioredoxin protein); then ion exchange (increase rhIFN α-2b purity).

### Immobilized Metal Affinity Chromatography (IMAC) Purification

A first IMAC column was used to purify the fusion thioredoxin-rhIFN α-2b protein from conditioned cell lysate. Fusion thioredoxin-rhIFN α-2b protein binds to an IMAC column due to the histidine content of thioredoxin. IMAC purification was performed using an ÄKTA Explorer 100 Chromatography System (GE Healthcare).

### Enzymatic Cleavage

Removal of the thioredoxin fusion protein, linker, and cleavage site was accomplished via enzymatic cleavage with enterokinase. A sample containing fusion protein was diluted with enterokinase cleavage buffer and incubated with enterokinase light chain enzyme for 16 hours at room temperature. An improvement was made to the cleavage step by first reducing the fusion protein with TCEP (tris(2-carboxyethyl)phosphine). The protein is then refolded spontaneously, or with the addition of copper and oxygen.

A second IMAC step purifies rhIFN α-2b protein after enzymatic cleavage from thioredoxin. The pH and conductivity of the enterokinase enzymatic cleavage sample (after cleavage) was verified and conditioned with IMAC equilibration buffer until a pH of 7.4±0.2 was achieved and the conductivity was no more than the conductivity of the equilibrium buffer (approximately 17 mS/cm).

Protein concentration of each fraction was measured with A280 measurements. Fractions were stored at 4° C. The rhIFN α-2b protein does not bind to the IMAC column and leaves the column in the load flow-through and wash. Other chromatography resins could be used, such as ion exchange and hydrophobic interaction chromatography. An optional third chromatography step could also be employed.

### Results

### First IMAC Purification

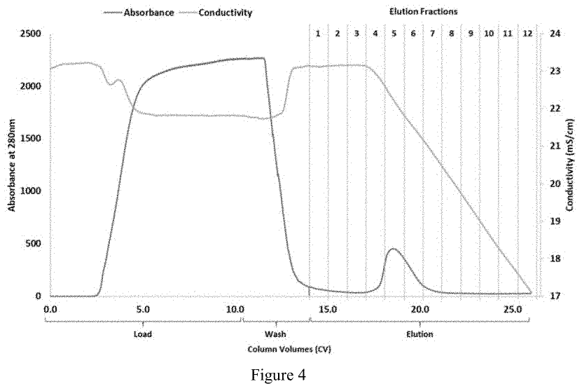

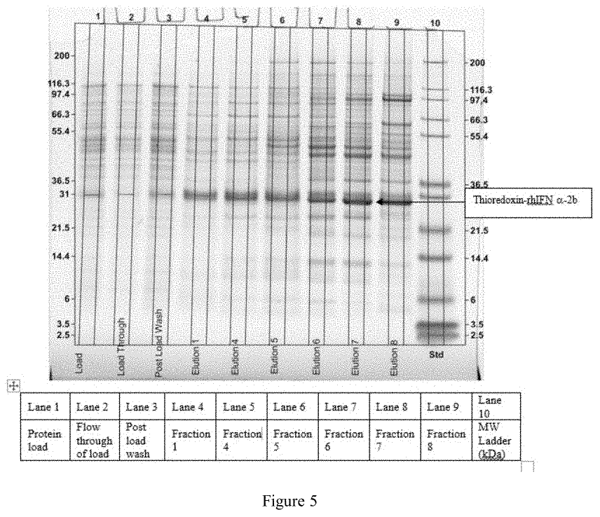

The ÄKTA chromatogram showing A280 and conductivity over the load, wash, and elution steps of the first IMAC purification is shown in FIG. 4. Breakthrough occurred at approximately three column volumes (CV), where the protein and other lysate components that were not bound to the column flowed through the column in the effluent. The absorbance increased during the breakthrough curve, then began to stabilize as the concentration of the effluent maximized at the feed concentration. The conductivity decreased during the loading step as the conductivity of the lysate load was less than that of the equilibration buffer. The conductivity decreased linearly during the gradient elution as the salt concentration decreased. The target protein was eluted from the column during the imidazole gradient as shown in the absorbance peak at approximately 18 CV. Material from the eluate of the first IMAC purification step was visualized with SDS-PAGE (FIG. 5).

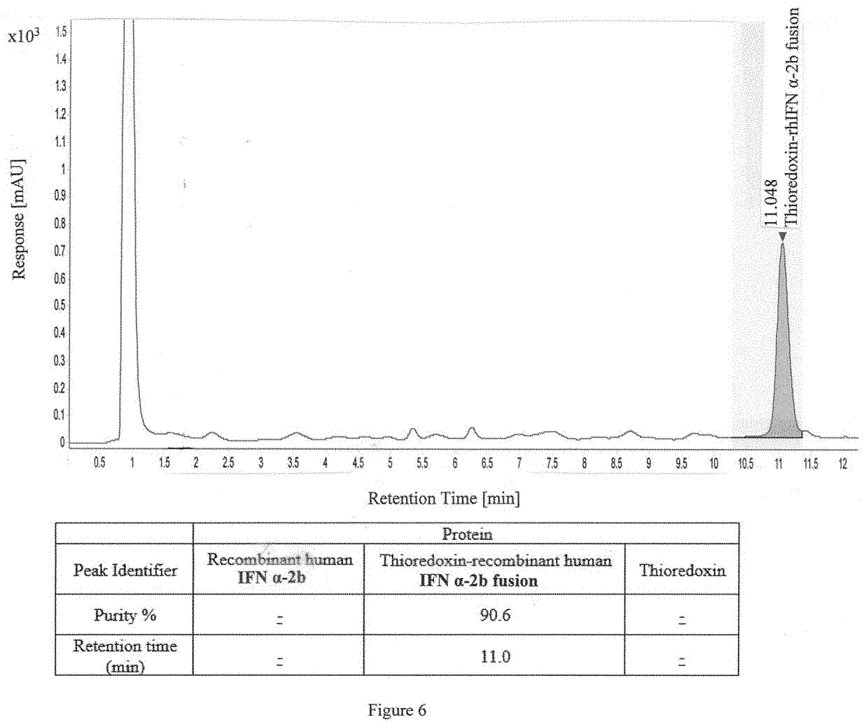

Elution fraction 7 from the first IMAC purification step was further analyzed with RP-HPLC (FIG. 6). This fraction was chosen due to the high concentration of thioredoxin-rhIFN α-2b fusion protein (determined by SDS-PAGE) within that fraction. The thioredoxin-rhIFN α-2b fusion protein was observed to have an approximate retention time of 11 minutes. Isoform peaks were not observed following the fusion protein peak.

### Enzymatic Cleavage

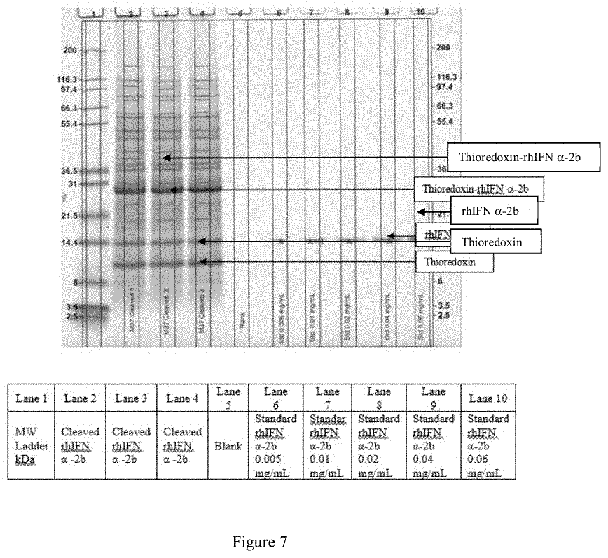

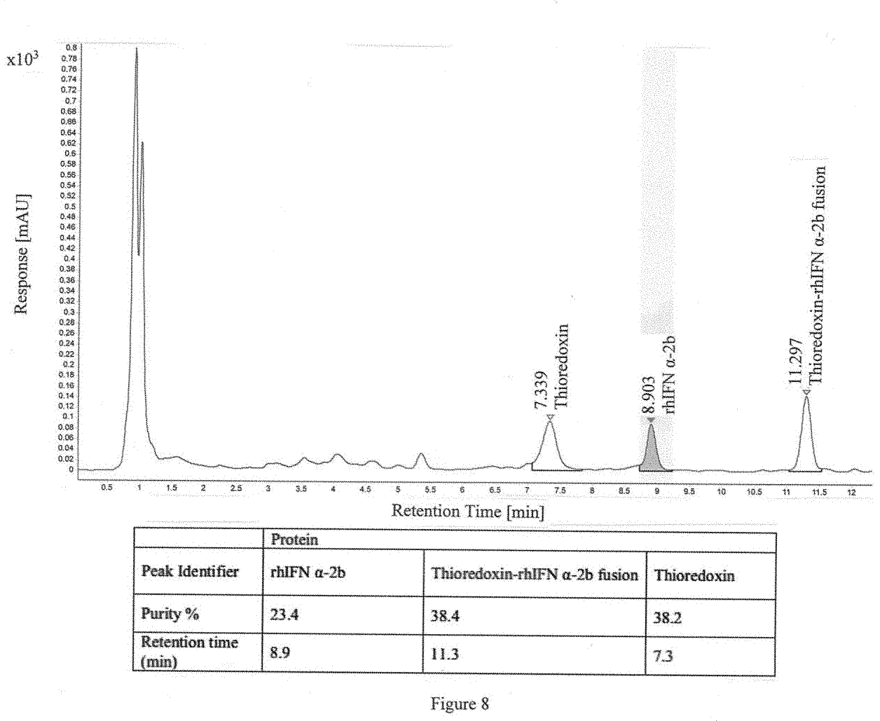

Enzymatic cleavage was evaluated with SDS-PAGE (FIG. 7) and RP-HPLC (FIG. 8). Separation of rhIFN α-2b from thioredoxin was observed via both analytical methods. Uncleaved thioredoxin-rhIFN α-2b fusion protein was also observed.

The cleaved rhIFN α-2b protein was observed to have an approximate retention time of 9 minutes, while the thioredoxin rhIFN α-2b fusion protein RT remained at approximately 11 minutes. The thioredoxin leader sequence was observed to have a RT of 7 minutes. The cleavage process could be optimized to yield approximately 40% rhIFN-α-2b and 60% thioredoxin and no fusion protein.

### Second IMAC Purification

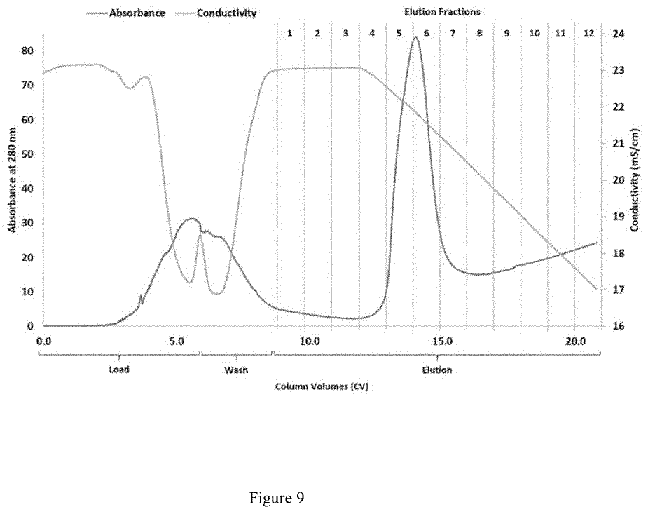

The AKTA chromatogram of the second IMAC purification showing A280 and conductivity over the load, wash, and elution steps is shown in FIG. 9. Breakthrough occurred at approximately three column volumes (CV), where the rhIFN α-2b target protein was observed in the flow-through. The absorbance continued to increase during the load and wash steps as the target protein continued to flow through the column and the uncleaved thioredoxin rhIFN α-2b fusion protein, thioredoxin leader sequence and other impurities were bound to the column. Absorbance decreased during the wash as the final target protein was washed off the column. The conductivity decreased during the loading step as the conductivity of the enzymatic cleavage reaction load was less than that of the equilibration buffer.

The conductivity increased to the conductivity of the equilibration buffer prior to the elution step. The conductivity decreased linearly during the gradient elution as the salt concentration decreased. The uncleaved thioredoxin rhIFN α-2b fusion protein, thioredoxin protein and other bound components were eluted from the column during the imidazole gradient as shown in the absorbance peak at approximately 14 CV. The absorbance signal increased slightly at the end of the gradient elution likely due to the increase of imidazole concentration in the eluent.

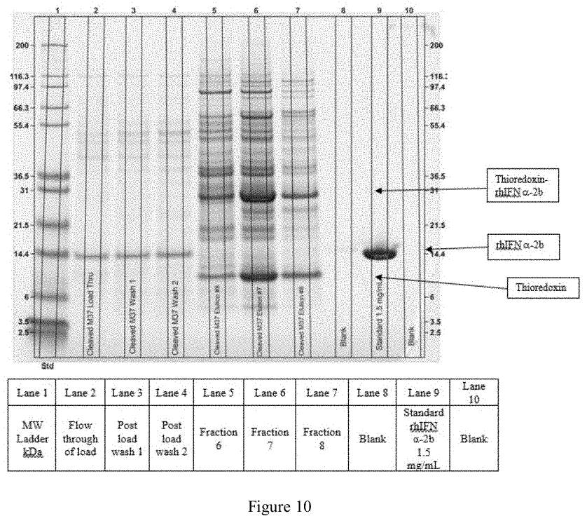

Material containing rhIFN α-2b was observed within the load flow-through and the wash for the second IMAC purification. This is expected as rhIFN α-2b would not be expected to bind to the IMAC column without the thioredoxin fusion protein. Thioredoxin and uncleaved thioredoxin-rhIFN α-2b was observed in the elution fractions (FIG. 10). Overall purity achieved, by RP-HPLC, was 100% relative to isoforms, thioredoxin and fusion protein. FIG. 10 also shows high purity in that only faint bands are found with the rhIFN α-2b in lanes 2, 3 and 4.

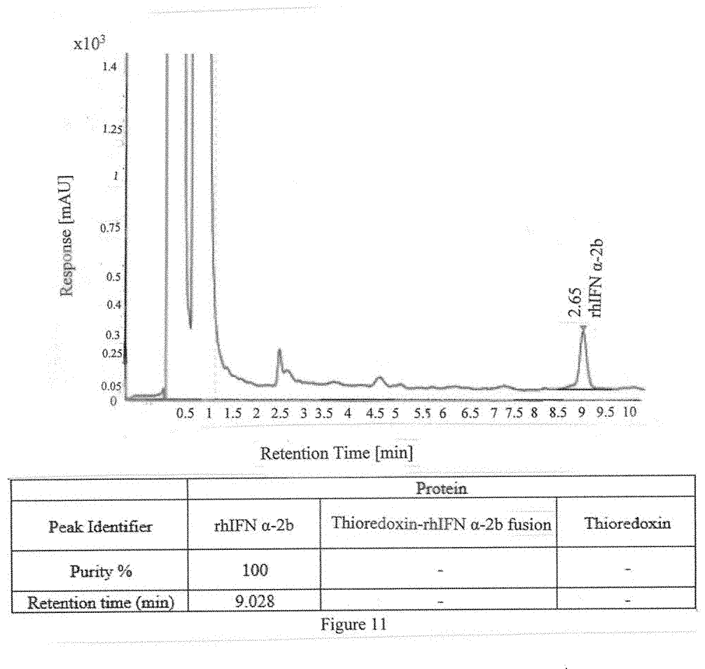

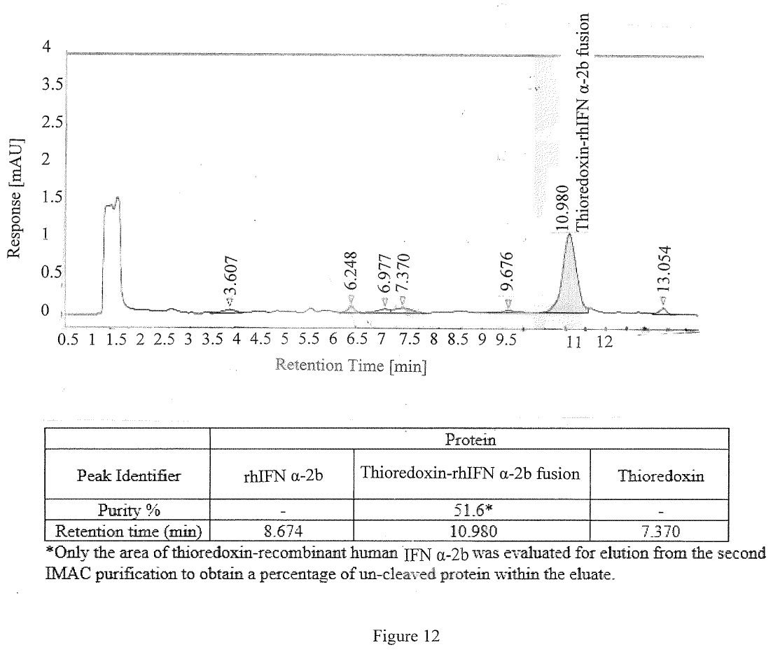

The cleaved rhIFN α-2b protein was observed to have an approximate RT of 9 minutes. Uncleaved thioredoxin-rhIFN α-2b and the thioredoxin leader sequence were not observed in the chromatogram at significant quantities relative to the target protein (FIG. 11). Analysis of elution fraction 7 by RP-HPLC was performed to quantify the percentage of uncleaved thioredoxin-rhIFN α-2b fusion protein within the elution (FIG. 12).

### Example Three

### Recombinant Human Isoform Free IFN α2B Formulations:

| | | Sodium | Sodium | | | | | | |
|---|---|---|---|---|---|---|---|---|---|
| | | Phosphate | phosphate | | | | | | |
| | | dibasic | monobasic | | | | | | |
| | | (hydration free | hydration | | Polysorbate | | D-ascorbic | | |
| | NaCl | weight) | free weight) | EDTA | 80 | Methionine | acid | Vitamin C | Dithiothreitol |
| F1 | 7.5 | 1.8 | 1.3 | 0.1 | 0.1 | 0 | 0 | 0 | 0 |
| F2 | 7.5 | 1.8 | 1.3 | 0.1 | 0.1 | 0 | 0.18 | 0 | 0 |
| F3 | 7.5 | 1.8 | 1.3 | 0.1 | 0.1 | 0 | 1.8 | 0 | 0 |
| F4 | 7.5 | 0 | 0 | 0.1 | 0.1 | 3.5 | 0 | 0 | 0 |
| F5 | 7.5 | 0.9 | 0.7 | 0.1 | 0.1 | 1.8 | 0 | 0 | 0 |
| F6 | 7.5 | 1.8 | 1.3 | 0.1 | 0.1 | 0 | 0 | 0.18 | 0 |
| F7 | 7.5 | 1.8 | 1.3 | 0.1 | 0.1 | 0 | 0 | 1.8 | 0 |
| F8 | 7.5 | 1.8 | 1.3 | 0.1 | 0.1 | 0 | 0 | 0 | 30.8 |

* all values mg/ml
* pure isoform free IFNα2b concentrations ranged from 10 to 400 μg/ml for each of the eight formulations.
* F1-F8 heterogeneous IFN reference standard (activity tested against)
* glutathione and lipoic acid were optionally added to each of the formulations at each concentration.
* other polysorbates (substituted) and poloxamers were further optionally added.

### Example Four

### rhIFN-α 2b Formulations

Pure isoform free human IFN α-2b is synthesized and provided in sterile vials in desired amounts such as for example from about 0.5 MIU to about 10 MIU per vial. Vials of various volume such as 0.5 ml or 1.0 ml are made (comprising buffer, antioxidant stabilizer agent). Liquid formulations can be frozen, or lyophilized and frozen; or reconstituted in sterile water to a desired concentrations. Reconstituted solution can be further diluted to make formulations of desired concentrations. Antiviral activity assays are used to test activity and determine activity units of interferon compared with standard interferon IC₅₀ (pM), IU/mol and relative bioactivity (molar basis). Biological activity of human recombinant IFN-alpha 2B can be measured with A549 cells exposed to the encephalomyocarditis (EMC) virus in a cytopathic effect (CPE) assay, cell survival measured using a fluorometric assay (Besnier et al., Proc Natl Acad Sci USA, 99(18)(2002) 11920-11925). The EC50 is defined as the effective concentration of the cytokine at which cell survival is at 50% of maximum.

The interferon unit or International unit for interferon (U or IU, for international unit) has been reported as a measure of IFN activity defined as the amount necessary to protect 50% of the cells against viral damage. A further assay that may be used to measure bioactivity is the cytopathic effect inhibition assay as described (Rubinstein, et al. 1981; Familletti, P. C., et al., 1981). In this antiviral assay for interferon about 1 unit/ml of interferon is the quantity necessary to produce a cytopathic effect of 50%. The units are determined with respect to the international reference standard for Hu-IFN-beta provided by the National Institutes of Health (Pestka, S. 1986).

The activity and determination of activity units of interferon is also currently determined by in vitro assay using the engineered cell lines HEK-Blue™ IFN-α/β, iLite™ Human Type I Interferon Responsive Cells. These cell lines have interferon inducible promoter(s) linked to a reporter gene. Activity units of interferon can be reported as IC₅₀, IU/mL or relative potency.

### Example Five

### Stability of hrIFN-α2b Formulations

Bulk solutions of human isoform free recombinant human IFN α-2b interferon formulations in accordance with the present invention were prepared. In order to provide a robust aqueous formulation of alpha interferon having sufficiently high concentrations of alpha interferon to provide a therapeutic dose, the formulations must be physically, chemically and functionally stable, as well as microbe free with endotoxin levels less than threshold level established as safe, i.e., less than 500 IU/mL.

Solutions of human recombinant IFN α-2b interferon in accordance with the present invention were tested at various time points: 0 days, 1 week, 2 weeks, 3 weeks, 4 weeks, 6 weeks, 2 months, 3 months, and 6 months. Statistical analysis was used to extrapolate stability beyond the 6 months. At each timepoint, using critical quality attributes (CQAs), RP-UHPLC, SEC-UHPLC and SDS-PAGE were performed. The pH was determined and appearance of formulations visually assessed. Storage condition temperatures of about −20° C.+/−5° C.; about 2° C.+/−3° C. to about 8° C.+/−3° C.; and about 25° C.+/−2° C. (60%+/−5% RH) and 40° C.+/−2° C. (75%+/−5% RH) were tested. A target pH was about 7.5+/−0.3 and osmolality measured.

As bulk formulations are shipped frozen, it is important they be stable to the freeze/thaw process. Accordingly, a process study was executed to investigate what role freezing rates and formulation variables had on bulk stability, and to obtain information on the robustness of the bulk solution to exaggerated processing conditions related to manufacture (e.g. multiple freeze/thaw cycling). Formulations comprising up to 5.0 mg/ml methionine as a stabilizing agent remained stable through 7-9 freeze/thaw cycles.

The following non-limiting clinical formulations exhibited stability:
(1) Formulation F2: 7.5 mg/ml NaCl, 1.8 mg/ml sodium phosphate dibasic, 1.3 mg/ml sodium phosphate monobasic, 0.1 mg/ml EDTA, 0.1 mg/ml Polysorbate 80 and 2.2 mg/ml lipoic acid.
(2) Formulation F3: 7.5 mg/ml NaCl, 0.1 mg/ml EDTA, 0.1 mg/ml Polysorbate 80 and 3.5 mg/ml methionine.
(3) Formulation F4: 7.5 mg/ml NaCl, 0.9 mg/ml sodium phosphate dibasic, 0.7 mg/ml sodium phosphate monobasic, 0.1 mg/ml EDTA, 0.1 mg/ml Polysorbate 80 and 1.8 mg/ml methionine.

### Example Six

### Inhalation Formulation COVID-19

The isoform free and pure rhIFN-α2b was formulated for inhalation for administration into the lungs. The stable rhIFN-α2b formulation doses 5 MIU of the rhIFN-α 2b of the invention via inhalation for use twice daily for up to 14 days.

Patients for study (N=>300) characterized as follows:

### Inclusion Criteria:

1. Age≥18 years with any of the following risk factors:
Pre-existing pulmonary disease
Chronic kidney disease
Poorly controlled diabetes (Hb A1c>7.6%)
Cardiovascular disease
Hypertension
Immunosuppression due to cancer or organ transplantation
HIV infection
Use of immunomodulating agent for treatment of autoimmune disease
SpO2 saturation<94% on room air
Respiratory rate>24
Hear rate>125
OR age≥55 with or without the above risk factors

2. COVID-19 demonstrated by PCR within 48 hours prior to or at study enrollment 3. Symptomatic of COVID-19 defined as either:
Fever≥100.4 F./38 C. or
Cough or
Dyspnea or
Chest pain

4. COVID-19 symptoms for <5 days

### Exclusion Criteria:

1. Patient who test positive for Influenze A or B

During treatment, symptoms and symptom progression monitored.

After treatment for up to 14 days, resolution of symptoms was determined from baseline, for 72 hours of: fever≤100.3 F./37.9 C., RR≤24, Sp O2≥94% on room air, dyspnea (none/mind), and cough (none/mild). The alternative was progression or no change in symptoms. 80% power to detect a 30% improvement.

Positive effects when stable rhIFN-α2b formulation is administered shortly after infection, that is, in early stage infection.

1.-89. (canceled)

90. Isoform free human recombinant interferon α-2b (rhIFN α-2b).

91. The isoform free rhIFN α-2b of claim 90, wherein the rIFN α-2b is isolated and at least 99% pure, at least 99.5% pure or 100% pure with respect to degradation isoforms.

92. The isoform free rhIFN α-2b of claim 91, wherein said rIFN α-2b is characterized by one or more of: a homogenous N-terminal; exhibits a stable confirmation relating to oxidative protein folding of cysteine residue(s); has a high specific activity; is soluble; and exhibits antiviral activity and increased biological activity relative to heterogeneous IFN-α 2b.

93. A composition comprising or consisting of the isoform free rhIFN α-2b of claim 92.

94. The composition of claim 93, comprising an acceptable carrier, excipient, diluent or mixtures thereof, and optionally wherein said composition is sterile.

95. A formulation comprising the composition of claim 94, formulated with a stabilizing agent, wherein the stabilizing agent functions as an antioxidant to prevent/minimize deoxidation of the rhIFN α-2b and maintain a folded conformation.

96. The formulation of claim 95, wherein the agent is water soluble and selected from the group consisting of methionine, cysteine, vitamin C (D-ascorbic acid), glutathione, lipoic acid, uric acid and combinations thereof.

97. The formulation of claim 96, wherein the agent is methionine and is present in an amount of up to about 5 mg/ml, up to about 1.0 mg/ml, up to about 1.5 mg/ml, about 1.8 mg/ml, up to about 2.0 mg/ml, up to about 2.5 mg/ml, up to about 3.0 mg/ml, up to about 3.5 mg/ml, up to about 4.0 mg/ml, up to about 4.5 mg/ml, or about 5.0 mg/ml.

98. The formulation of claim 96, wherein the agent is lipoic acid and is present in an amount of up to about 1.0 mg/ml, up to about 1.5 mg/ml, about 1.8 mg/ml, up to about 2.0 mg/ml, about 2.2 mg/ml, up to about 2.5 mg/ml, up to about 3.0 mg/ml, up to about 3.5 mg/ml, up to about 4.0 mg/ml, up to about 4.5 mg/ml, or about 5.0 mg/ml.

99. The formulation of claim 95, wherein the rhIFN α-2b is provided in a desired concentration per ml or in an amount of up to about 10 mg/ml; up to about 15 mg/ml; at about 10 μg/ml to about 400 μg/ml; or at about 5.0 MIU/ml.

100. The formulation of claim 95, wherein the formulation is made from a reconstituted lyophilized composition of claim 93.

101. The formulation of claim 95, wherein the formulation is stable at temperatures ranging from about −80° C. to about 40° C. for up to about 1 month, up to about 2 months, up to about 3 months, up to about 6 months, up to about 1 year or up to about 2 years.

102. The formulation of claim 95, wherein the formulation is stable at temperatures ranging from about 2° C. to about 40° C. for several months.

103. The formulation of claim 95, formulated as a liquid for administration via inhalation, intravenous, intramuscular, intrathecal, parenteral or intravaginal, and wherein the liquid is a liquid aerosol, liquid droplets, liquid mist or as a liquid spray.

104. The formulation of claim 103, formulated for nasal inhalation and/or lung inhalation.

105. The formulation of claim 104, in conjunction with a metered dose inhaler (MDI), soft mist inhaler (SMI), nebulizer, spray bottle or droplets bottle.

106. The formulation of claim 95, formulated for intravaginal administration as an aerosol, as droplets, as a mist, as a spray, as a gel or as a creme.

107. The formulation of claim 95, formulated as a dry powder, as a powder aerosol, a powder mist, or a powder particulate spray, wherein the dry powder comprises particles of up to about 5 μm and optionally provided in conjunction with a dry powder inhaler (DPI) or dry powder metered dose inhaler.

108. The formulation of claim 95, further comprising or used in conjunction with one or more of: an antiviral selected from Remdesivir, lopinavir, Favipiravir, Darunavir, Oseltamivir, Umifenovir and Novaferon; an immune modulating drug selected from immunoglobulins and monoclonal antibodies; a glucocorticoid; an anti-inflammatory drug selected from leflunomide, colchicine, naproxen and piclidenoson; a cardiovascular drug selected from ACE-2, ACE-inhibitor, ARB and angiotension; Chloroquine; Hydroxychloroquine; Aviptadil; Azithromycin; Itraconazole; Vitamin D; and zinc.

109. The formulation of claim 95 comprising:
about 10 μg/ml to about 400 μg/ml of said isoform free rhIFN α-2b;
up to about 5 mg/ml methionine;
Polysorbate 80;
EDTA; and
sodium phosphate buffer,
wherein said formulation is stable at temperatures ranging from about −80° C. to about 40° C. and/or said formulation is stable at temperatures of about 2° C. to about 40° C. for up to six months.

110. A method of treating a viral respiratory infection in a subject, comprising administering by inhalation to the respiratory tract of the subject and/or inhalation via nasal passages, an effective amount of the formulation of claim 104.

111. The method of claim 110, wherein the viral respiratory infection is caused by a virus selected from:
a coronavirus (alpha, beta, gamma and delta); human coronaviruses 229E (alpha coronavirus), NL63 (alpha coronavirus), OC43 (beta coronavirus), and HKU1 (beta coronavirus); MERS-CoV (beta coronavirus causing Middle East respiratory syndrome); SARS-CoV (beta coronavirus causing severe acute respiratory syndrome); and SARS-CoV-2 (new coronavirus causing COVID-19 disease); or
an influenza virus (swine flu H1N1 influenza-A virus; avian flu H5N1 influenza-A virus; seasonal influenza A or B) a parainfluenza virus, an adenovirus (common cold), respiratory syncytial virus (RSV), a rhinovirus or a meta-pneumovirus.

112. The method of claim 111, wherein the virus is SARS-CoV-2 causing COVID-19 disease.

113. The method of claim 112, wherein said formulation is provided at a dose of up to about 5 MIU/ml for twice daily administration for up to about 14 days.

114. The method of claim 113, wherein the formulation is for administration to the subject with a first symptom of COVID-19 disease, prior to a first symptom of COVID-19 disease or at risk of infection with SARS-CoV-2.

115. A formulation of isoform free recombinant human interferon alpha 2b (rIFN α-2b), wherein said formulation is selected from:
(a) a formulation comprising up to about 10 mg/ml rhIFN-α 2b, about 7.5 mg/ml NaCl, about 1.8 mg/ml sodium phosphate dibasic, about 1.3 mg/ml sodium phosphate monobasic, about 0.1 mg/ml EDTA, about 0.1 mg/ml Polysorbate 80 and about 2.2 mg/ml lipoic acid;
(b) a formulation comprising up to about 10 mg/ml rhIFN α-2b, about 7.5 mg/ml NaCl, about 0.1 mg/ml EDTA, about 0.1 mg/ml Polysorbate 80 and about 3.5 mg/ml methionine;
(c) a formulation comprising up to about 10 mg/ml rhIFN α-2b, about 7.5 mg/ml NaCl, about 0.9 mg/ml sodium phosphate dibasic, about 0.7 mg/ml sodium phosphate monobasic, about 0.1 mg/ml EDTA, about 0.1 mg/ml Polysorbate 80 and about 1.8 mg/ml methionine;
(d) a formulation comprising up to about 10 mg/ml rhIFN α-2b, up to about 10 mg/ml NaCl, up to about 5.0 mg/ml sodium phosphate dibasic, up to about 5.0 mg/ml sodium phosphate monobasic, about to about 3.0 mg/ml EDTA, up to about 3.0 mg/ml Polysorbate 80 and up to about 4.0 mg/ml lipoic acid; and
(e) a formulation comprising up to about 10 mg/ml rhIFN α-2b, up to about 10.0 mg/ml NaCl, up to about 4.0 mg/ml EDTA, up to about 3.0 mg/ml Polysorbate 80 and up to about 5.0 mg/ml methionine.

116. The formulation of claim 115, wherein the formulation has a pH of about 7.2 to about 7.8 and is stable at freezing, refrigerated and room storage temperatures for at least several months.


SEQUENCE LISTING (15 sequences)

SEQ ID NO: 1 - protein, 166 aa, Homo sapiens [sequence omitted]

SEQ ID NO: 2 - protein, 247 aa, Artificial Sequence
  Synthetic
MHHHHHHQIFVKTLTGKTITLEVEPSDTIENVKAKIQDKEGIPPDQQRLIFAGKQLEDGRTLSDYNIQKESTLHLVLRLRGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 284 aa, Artificial Sequence
  Synthetic
MSDKIIHLTDDSFDTDVLKADGAILVAFWAHWCGPCKMIAPILDEIADEYQGKLTVAKLNIDHNPGTAPKYGIRSIPTLLLFKNGEVAATKVGALSKGQLKEFLDANLAGSGSGDDDDKCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 4 - protein, 251 aa, Artificial Sequence
  Synthetic
MSDKIHHHHHHQIFVKTLTGKTITLEVEPSDTIENVKAKIQDKEGIPPDQQRLIFAGKQLEDGRTLSDYNIQKESTLHLVLRLRGGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 5 - protein, 10 aa, Artificial Sequence
  Synthetic
GSGSGDDDDK

SEQ ID NO: 6 - protein, 4 aa, Artificial Sequence
  Synthetic
GSEQ

SEQ ID NO: 7 - protein, 5 aa, Artificial Sequence
  Synthetic
GSDEE

SEQ ID NO: 8 - protein, 9 aa, Artificial Sequence
  Synthetic
GSEEEDDDG

SEQ ID NO: 9 - protein, 11 aa, Artificial Sequence
  Synthetic
GSEEEDDDGKK

SEQ ID NO: 10 - protein, 13 aa, Artificial Sequence
  Synthetic
GSEQKGGGEEDDG

SEQ ID NO: 11 - protein, 10 aa, Artificial Sequence
  Synthetic
GSEEDDDEEK

SEQ ID NO: 12 - protein, 14 aa, Artificial Sequence
  Synthetic
GSEQKGGGEEDDEE

SEQ ID NO: 13 - protein, 12 aa, Artificial Sequence
  Synthetic
GSEQKGGGDEDG

SEQ ID NO: 14 - protein, 165 aa, Artificial Sequence [sequence omitted]

SEQ ID NO: 15 - protein, 170 aa, Artificial Sequence
  Synthetic
DDDDKCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE